(19) United States
(12) Patent Application Publication
(10) Pub. No.: US 2004/0097408 A1
(43) Pub. Date: May 20, 2004

(54) Compounds regulating cell proliferation and differentiation

(76) Inventors: Philip Leder, Chestnut Hill, MA (US); Valeria R. Fantin, Boston, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/613,762

(22) Filed: Jul. 3, 2003

Related U.S. Application Data
(63) Continuation of application No. PCT/US02/00307, filed on Jan. 3, 2002.
(60) Provisional application No. 60/259,444, filed on Jan. 3, 2001.
(60) Provisional application No. 60/297,739, filed on Jun. 12, 2001.

(51) Int. Cl. A61K 38/00

(52) U.S. Cl. 514/2

(57) ABSTRACT

The invention provides compounds and methods for normalizing the proliferation and/or modulating differentiation and/or inducing the cell death of cells. In a preferred embodiment, the invention provides methods for inhibiting proliferation of hyper-proliferative cells, comprising contacting the cells with a composition comprising a growth inhibiting amount of one or more compounds selected from the group consisting of E20 (ID 141525); F12 (ID 120590); F16 (ID 274873); H10 (ID 120670); J6 (ID 120856); N12 (ID 215015); L4 (ID 121113); B 15 (ID 217496) and QR, or derivatives or analogs thereof, or pharmaceutically acceptable salts thereof.

### CROSS-REFERENCE TO RELATED APPLICATIONS

This application claims the benefit of International Application No. PCT/US02/00307, filed Jan. 3, 2002 and published in English under PCT article 21(2), which claims the benefit of U.S. Provisional Application No. 60/259,444, filed Jan. 3, 2001 and U.S. Provisional Application 60/297,739, filed Jun. 12, 2001. These applications are specifically incorporated by reference herein.

### BACKGROUND OF THE INVENTION

Normal tissue homeostasis is achieved by an intricate balance between the rate of cell proliferation and cell death. Disruption of this balance either by increasing the rate of cell proliferation or decreasing the rate of cell death can result in the abnormal growth of cells and is thought to be a major event in the development of cancer, as well as other cell proliferative disorders such as restenosis.

The effects of cancer are catastrophic, causing over half a million deaths per year in the United States alone. Conventional strategies for the treatment of cancer include chemotherapy, radiotherapy, surgery or combinations thereof, however further advances in these strategies are limited by lack of specificity and excessive toxicity to normal tissues. In addition, certain cancers are refractory to treatments such as chemotherapy, and some of these strategies such as surgery are not always viable alternatives. For example, non-small-cell lung cancer (NSCLC), which includes squamous cell carcinoma, adenocarcinoma and large-cell carcinoma, accounts for 75-80% of all lung cancers (American Cancer Society, 1993). Current multimodality therapeutic strategies applied to regionally advanced NSCLC are minimally effective with the overall cure rate being only about 10% (Belani et al. (1993) Semin. Oncol. 20:302 and Roth et al. (1994) Lung Cancer 11 Suppl. 3:S25).

Cancer is now understood to be the result of multiple genetic changes (Goyette et al. (1992) Mol. Cell Biol. 12:1387) and it is well established that many cancers are caused, at least in part, by genetic alterations that result in either the over expression of one or more genes, or the expression of abnormal or mutant gene or genes. For example, the expression of oncogenes is known to play a role in the development of cancer. Oncogenes are defined as genetically altered genes whose mutated expression product or level of expression somehow disrupts normal cellular function or control (Spandidos et al. (1989) Anticancer Res. 9:821). These types of mutations are believed to have effects on the malignant growth of cells derived from practically every tissue. Oncogenes also include tumor suppressor genes, such as p53 and p53-like genes, whose lack of expression results in uncontrolled cell proliferation.

The Neu/HER-2/erbB2 proto-oncogene is a member of the epidermal growth factor (EGF) receptor family. Neu is a rodent gene and Her-2 is its human homologue. This group of receptor tyrosine kinases plays an essential role during growth and differentiation of many tissues. In addition, overexpression of these proteins is associated with several types of human cancers (reviewed in Hynes et al. (1994) Biochim Biophys Acta 1198(2-3), 165-84; Kim et al. (1999) Exp Cell Res 253(1), 78-87; and Hung et al. (1999) Seminars in Oncology 26 (4 Suppl 12), 51-9). The Neu/Her-2 gene is amplified and the protein overexpressed in 20-30% of human breast carcinomas, and this event correlates with poor prognosis. This observation strongly suggests that Neu/Her-2 plays a direct role in the development of breast tumors. Furthermore, targeted expression of constitutively active Neu to the mouse mammary gland results in induction of multifocal mammary tumors in females harboring the transgene (Muller et al. (1988) Cell 54(1), 105-15; Andrechek et al.(2000) PNAS 97(7), 3444-9). In addition, Neu antisense treatment not only affects proliferation but also activates apoptotic pathways in Neu-overexpressing cells, demonstrating that this proto-oncogene is involved in both proliferation and survival (Roh et al. (2000) Cancer Res 60(3), 560-5).

The fact that repression of Neu tyrosine kinase suppresses the malignant phenotype of cells overexpressing the oncogene together with its extracellular accessibility have made Neu an excellent target for tumor therapy. For that reason, efforts have been directed towards the development of reagents that directly interfere with Neu activity. Firstly, small molecules that inhibit the tyrosine kinase activity of Neu are being developed. Tyrphostins are an example of this category of compounds (Levitzki et al. (1995) Science 267(5205), 1782-8). Inhibitory activity towards a vast array of protein tyrosine kinases in the nanomolar and micromolar range has been reported for several tyrphostins in vitro, but only a few work on intact cells. More recently AG-1478, a quinazoline inhibitor of the EGF receptor tyrosine kinase, has been shown to suppress tumorigenesis in MMTV/Neu+TGFα bigenic mice. This compound induces the sequestration of Neu in inactive Neu/EGF receptor heterodimers (Lenferink et al. (2000) PNAS 97(17), 9609-14). AG1478 and PD153035 both bind to the ATP binding site on the EGF receptor, acting as competitive inhibitors. These compounds have a cytostatic effect on cells, i.e. cells will continue to proliferate upon removal of these drugs (Albanell et al. (1999) Journal of Mammary Gland Biology & Neoplasia 4(4), 337-51). One of the major drawbacks of these types of inhibitors is that, in general, they affect more than one tyrosine kinase due to the high degree of homology within certain conserved domains of this class of enzymes (Levitzki et al. (1995) Science 267(5205), 1782-8). Thus, the major challenge at the moment lies in the improvement of selectivity for several of these promising compounds. In addition, the anti-proliferative effect of natural tyrosine kinase inhibitors, and a new class of irreversible inhibitors of Neu are currently being tested on Neu-overexpressing cancer cells (Hung et al. (1999) Seminars in Oncology 26(4 Suppl 12), 51-9; Arteaga et al. (1997) J Biol Chem 272(37), 23247-54; and Fry et al.(1998) Proc Natl Acad Sci USA 95(20), 12022-7).

Another approach to inhibiting Neu is the use of neutralizing monoclonal antibodies directed against the extracellular domain of Neu. Herceptin™, the humanized recombinant version of 4D5, has shown in general an anti-proliferative effect on Neu-overexpressing cells, and have proven successful when used in combination with cytotoxic chemotherapeutic agents for the treatment of Neu-overexpressing breast cancers (Hudziak et al.(1989) Mol Cell Biol 9(3), 1165-72; Carter et al. (1992) Proc Natl Acad Sci USA 89(10), 4285-9; Lewis et al.(1993) Cancer Immunol Immunother 37(4), 255-63; Pegram et al. (1999) Oncogene 18(13), 2241-51; and Pegram et al. (1998) Breast Cancer Research & Treatment 52(1-3), 65-77). Although treatment of cells expressing high levels of Neu with neutralizing antibodies results in a decrease in receptor phosphorylation and Neu protein level, not all cells are growth inhibited (Lane et al. (2000) Mol Cell Biol 20(9), 3210-23). The outcome from clinical studies performed on patients with tumors overexpressing Neu treated with Herceptin™ is that not all respond to the antibody treatment (Cobleigh et al. (1999) Journal of Clinical Oncology 17(9), 2639-48). These results correlate with the observations at the cellular level, and suggest that other pathways might be contributing to the uncontrolled growth of Neu-overexpressing tumor cells.

Thus, there is a need for identifying compounds which are effective at low doses in inhibiting excessive cell proliferation or stimulating differentiation or killing cells proliferating excessively, and which preferably have reduced cytotoxicity towards normal cells.

### SUMMARY OF THE INVENTION

The invention provides methods and compositions for inhibiting proliferation and/or modulating differentiation of cells, or for killing cells (referred to herein as “target cells”). In a preferred embodiment, the target cells are cells which proliferate excessively, such as cells which have a defect in the normal mechanism of growth control or differentiation. For example, the cells may be cells which are defective in their response to a growth factor, e.g., epidermal growth factor (EGF). The defect may be located in a receptor or in a signal transduction molecule. The defect may be caused by the presence of an oncogene. For example, the invention provides methods and compositions for inhibiting cell proliferation and/or modulating cell differentiation or killing cells that express constitutive forms of receptor kinases, e.g., receptor tyrosine kinases, such as Neu. Other preferred target cells are those which express a constitutively active form of Ras or Myc. Yet other target cells are cells having a high mitochondrial membrane potential (Ψm) relative to most normal cells.

Preferred compounds of the invention inhibit cell proliferation and/or modulate cell differentiation or kill cells proliferating excessively, essentially without affecting cells which proliferate normally. For example, certain compounds of the invention inhibit proliferation of transformed cells, such as cells tranformed by an oncogene, but do not significantly affect the proliferation of counterpart non-transformed cells.

In general, the compounds of the present invention are small molecules that comprise an aromatic moiety, e.g., optionally substituted phenyl, and an aromatic or non-aromatic heterocyclic moiety, e.g., an optionally substituted nitrogen-containing heterocyclyl. In certain embodiments, the compounds of the invention are represented by structures A, B C, or D, wherein the definitions of their various substituents are presented in the Detailed Description of the Invention.

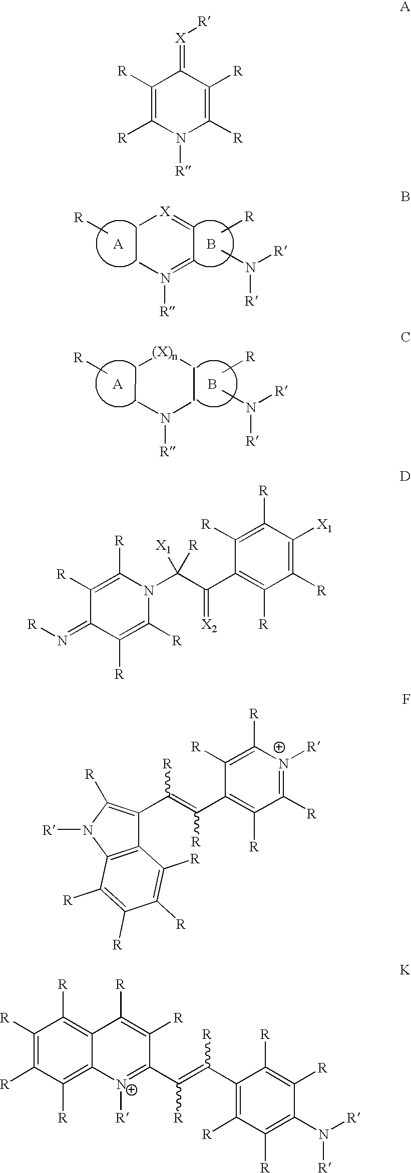

Preferred compounds are those having the general formula E, F, G, H, I, J or K. Even more preferred compounds are E20 (ID 141525); F12 (ID 120590); F16 (ID 274873); H10 (ID 120670); J6 (ID 120856); N12 (ID 215015); L4 (ID 121113); B15 (ID217496) and QR or derivatives, analogs or prodrugs thereof that are capable of inhibiting cell proliferation and/or modulating cell differentiation or which are capable of killing cells.

The invention provides pharmaceutical compositions or preparations, comprising amounts of one or more compounds having the general formula A, B, C, D, E, F, G, H, I, J or K, or pharmaceutically acceptable salts thereof and a pharmaceutical carrier or excipient. The effective amount can be an amount effective to inhibit cell proliferation, to modulate (e.g, inhibit) cell differentiation or induce cell death. Preferred pharmaceutical compositions comprise one or more compounds selected from the group consisting of E20 (ID 141525); F12 (ID 120590); F16 (ID 274873); H10 (ID 120670); J6 (ID 120856); N12 (ID 215015); L4 (ID 121113); B15 (ID217496) and QR, or derivatives, analogs or prodrugs thereof, or a pharmaceutically acceptable salt thereof.

The invention provides methods for treating and preventing conditions associated with an undesirable cell proliferation or differentiation. In one embodiment, the condition is a proliferative disease or condition, i.e., a disease or condition associated with an undesirable cell proliferation, e.g., benign or malignant cancer. Cancers that can be treated according to the method of the invention include those which are caused by, or associated with, expression of an activated form of an oncogene, e.g., the Neu proto-oncogene, such as breast and ovarian cancer, or with an activated form of the Ras proto-oncogene, such as epithelial cancers, e.g., prostate cancer, lung cancer, breast cancer, colorectal cancer, and pancreatic cancer. Other conditions that can be treated according to the invention involve non cancerous cell growth, e.g, cell proliferation resulting from a viral infection. Other conditions that can be treated according to the invention include diseases associated with a high Ψm.

In a preferred embodiment of the invention, the method comprises administering to a subject in need thereof, a pharmaceutically effective amount of a composition comprising one or more compounds having the general structure A, B, C, D, E, F, G, H, I, J or K, or a pharmaceutically acceptable salt thereof, e.g., a composition comprising one or more compounds selected from the group consisting of E20 (ID 141525); F12 (ID 120590); F16 (ID 274873); H10 (ID 120670); J6 (ID 120856); N12 (ID 215015); L4 (ID 121113); B15 (ID 217496) and QR, or derivatives, analogs or prodrugs thereof, or a pharmaceutically acceptable salt thereof. The compounds can be administered, e.g., orally or by injection. One or more compounds can be administered to a subject in need thereof. The compounds can also be administered to a subject together with other treatments, e.g., radiation therapy or chemotherapy. Where a compound is administered with another compound, these can be administered together or sequentially. A treatment protocol may consist in the administration of one or more doses of the compound of the invention.

The invention further provides kits comprising one or more of the compounds of the invention and optionally, instructions for use and/or other components.

Also within the scope of the invention are screening assays for identifying other compounds for use according to the invention.

At least one advantage of the compounds of the invention is their selectivity for cells which proliferate and/or differentiate in an unwanted manner, and the absence of significant effect on counterpart normal cells. For example, certain compounds of the invention inhibit substantially only the proliferation of transformed cells, and not that of their normal or wild-type counterpart. Thus, as opposed to most treatments for proliferative diseases, and in particular cancer, which kill both cancer cells and normal cells in a subject, the methods of the invention permit the selective targeting of transformed cells without significantly affecting non-transformed cells.

At least one other advantage of the compounds of the invention is that effectiveness in a range of very low concentrations. Preferred compounds of the invention are active at concentrations as low as 100 nM or even 10 nM. Such low concentrations reduce the likelihood of the compounds to negatively affect other cellular functions.

### BRIEF DESCRIPTION OF THE DRAWINGS

FIG. 1A shows the result of a cytoblot assay for BrdU incorporation in EpH4; EpH4-A6 and dEpH4-A8 cells exposed to 0, 2, or 20 μg/ml of anti-Neu antibody.

FIG. 1B shows one set of plates (shown in duplicate) out of the 51 sets tested in the high-throughput screen using a cytoblot assay for BrdU incorporation in EpH4-EV and EpH4-A6 cells exposed to anti-proliferative compounds.

FIGS. 2A-H show structures and proton NMR spectra of the compounds E20 (ID 141525); F12 (ID 120590); F16 (ID 274873); H10 (ID 120670); J6 (ID 120856); and N12 (ID 215015), respectively, which were shown to have anti-proliferative activity.

FIG. 3 shows the result of anti-BrdU cytoblot assays in which EpH4-A6 cells were exposed to concentrations from 100 to 0.01 μM of compound F12 (ID 120590), H10 (ID 120670), J6 (ID 120856), N12 (ID 215015), E20 (ID 141525) and F16 (ID 274873). F12, H10, J6 and N12 retain anti-proliferative activity up to 500 nM and F16 retains anti-proliferative activity up to 100 nM.

FIG. 4 shows the result of anti-BrdU cytoblot assays in which anti-proliferative small molecules F12, H10, J6, N12, E20, F16, L4, and B15 (compounds that score positive for EpH4-A6 cells at 10 μM) were incubated on a panel of tumor cell lines from neu, ras and myc initiated tumors.

FIG. 5 represents graphs showing the amount of BrdU incorporation and the percentage of cells in G1, S and G2 phase of the cell cycle after incubation of EpH-A6 and EpH4-EV cells with or without F16.

FIG. 6 represents the results of flow cytometry analysis of propidium iodide stained EpH4-A6 and EpH4-EV cells (grey and black columns, respectively) subjected to a time course treatment with F16.

FIG. 7 shows the level of F16 in serum of mice injected with 12.5 mg/kg or 25 mg/kg of F16 at 0, 15 minutes, 30 minutes, one hour, 3½ hours, 7 hours or 20 hours after the injection. The left hand column is a standard showing the signal obtained with known amounts of F16.

FIG. 8 shows two graphs (one for experiment 1 and one for experiment 2) of the weight of the tumors of the right and left flanks of nude mice injected with EpH-A6 and with F16 (F16-R and F16-L) or with vehicle (P-R and P-L).

FIG. 9 represents the results of a FACS analysis showing the amount of F16 in tumors of F16 treated- or placebo-treated nude mice in which EpH-A6 nude mice were injected.

FIG. 10 shows ATP levels in F16-treated EpH4-A6 cells (black columns); control cells (grey columns); and control FCCP-treated A6 cells (hatched column).

FIG. 11 shows flow cytometry analysis of ethidium bromide DNA fluorescence resulting from dihydroethidium oxidation as a measure of superoxide levels.

FIG. 12 shows Western blots of lysates from EpH4-A6 and EpH4-EV cells treated for 0, 18 or 36 hours with 3 μM F16, incubated with anti-pNeu; anti-βactin; andi-pPKB; anti-PKB; anti-pMAPK and anti-ERK-1 antibodies.

### DETAILED DESCRIPTION OF THE INVENTION

Overview

The invention is based at least in part on the identification of several small molecules, which inhibit proliferation of transformed cells, without substantially affecting the proliferation of non-transformed cells. For example, it has been found that the following compounds inhibit proliferation of, or kill, cells over-expressing Neu and/or Ras and/or Myc at very low concentrations, at about 1 μM or even 100 nM and 10 nM: E20 (ID 141525); F12 (ID 120590); F16 (ID 274873); H10 (ID 120670); J6 (ID 120856); N12 (ID 215015); L4 (ID 121113); B15 (ID 217496) and QR. These particular compounds constitute individual species of three generic classes of compounds, i.e., the classes of compounds represented by A, B, C, D, F and K, wherein their substituents are defined infra, that are expected to inhibit proliferation of transformed cells, without substantially affecting the proliferation of non-transformed cells. In certain embodiments, the present invention contemplates the use of any member or members of the afore mentioned classes of compounds in a method of the present invention.

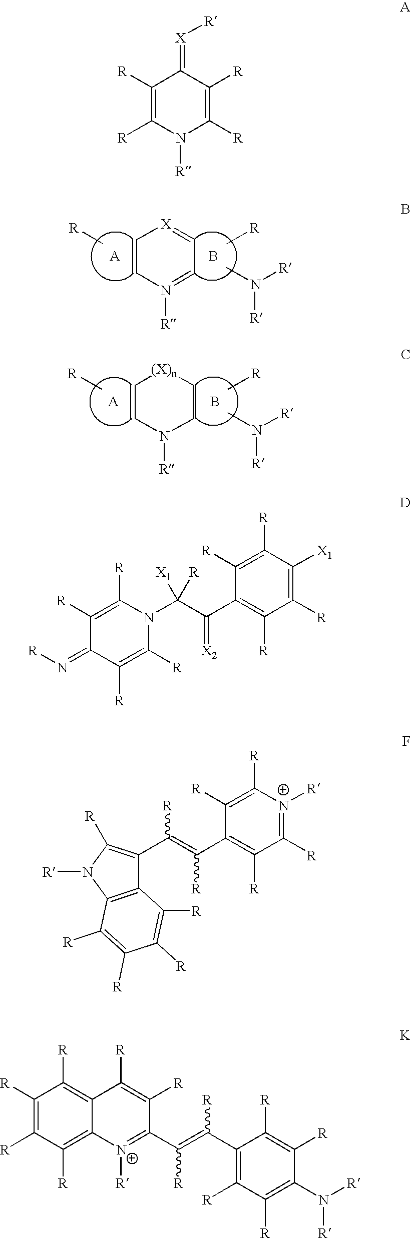

Briefly, in an attempt to find novel tools to selectively interfere with the progression of Neu overexpressing tumors, a chemical library was screened using a cell-based assay. For that purpose, constitutively active Neu was transfected into EpH4 mouse mammary epithelial cells (Niemann et al.(1998) J Cell Biol 143(2), 533-45). Control and stably transfected cells were screened in parallel. This isogenic system enabled the search for compounds that specifically inhibit cell proliferation dependent on Neu responses in a biological relevant environment. At the same time, this strategy allowed to discriminate compounds with elevated cytotoxicity towards non-transformed cells.

Since the compounds identified and described herein inhibit proliferation of Neu- and/or Ras- and/or Myc-transformed cells, these compounds are useful generally for inhibiting proliferation of cells having a defect in the same signal transduction pathways as those in which Neu and/or Ras and/or Myc are involved, or in complementary pathways. Since different signal transduction pathways may interact in cells, the compounds of the invention may be used to modulate other signal transduction pathways than those in which these particular oncogenes are involved. Thus, the compounds of the invention have a wide range of uses, and their effect on a particular signal transduction pathway or a particular type of cell can be ascertained by methods further described herein.

For example, based at least on the fact that Neu and Ras are generally involved in signal transduction in response to growth and differentiation factors, the compounds of the invention can be used to inhibit proliferation of cells having a defect in a response to a growth or differentiation factor. For example, the compounds of the invention can be used to regulate the proliferation of cells having an abnormal response to a growth factor, such as a growth factor selected from the group consisting of Epidermal Growth Factor (EGR); Fibroblast Growth Factor (FGF); Platelet Derived Growth Factor (PDGF); colony stimulating factors, such as Macrophage Colony Stimulating Factor (M-CSF); Nerve Growth Factor (NGF); Stem Cell Factor (SCF); interleukins such as IL-2, IL-3, IL-7. The cells responsive to the compounds of the invention (referred to as “responsive cells” or “target cells”) may have a defect in any of the signal transduction molecules (and growth factor receptors) in these pathways. Thus, for example, responsive cells may have a defect in a Src family kinase, e.g., Fyn, Lyn and Lck; in Sos; Ash/Grb2; Raf; MAP kinase kinase (MAPKK or MEK); or MAP kinase (MAPK). Responsive cells may also have a defect in transcription factors responding to the signal from such signal transducing molecules, e.g., in Fos and Jun. The term “defect” in a molecule, e.g., a protein, such as a cell surface receptor or a signal transduction molecule, refers to a mutation in the gene, resulting in expression of a mutated form of the protein or over-or under-expression of the protein. For example, a defect in the Ras proto-oncogene or in the Neu proto-oncogene can be a point mutation resulting in a constitutively active form of the protein.

Since the above-described signal transduction molecules are involved in normal cell proliferation, the compounds of the invention can also be used to normalize cell proliferation of cells which are not cancerous, but which are proliferating undesirably, e.g., abnormally due to another cause. For example, the compounds can be used to inhibit proliferation of virally infected cells, e.g., cells infected with a human papilloma virus (HPV). The compounds can also be used to reduce cell proliferation resulting from the presence of abnormally high concentrations of certain growth or differentiation factors. Thus, instead of neutralizing such growth or differentiation factor as a method of treatment, one or more compounds of the invention could be administered to the subject.

In view of the connection between cell proliferation and cell differentiation, the compounds of the invention can also be used for modulating cell differentiation. Generally, inhibition of proliferation correlates with increased differentiation. Accordingly, in certain embodiments of the invention, cell differentiation is stimulated by contacting cells with a compound of the invention.

Certain of the compounds of the invention are useful for inducing cell death, e.g., by apoptosis, as opposed to arresting cell cycle as other compounds of the invention do (i.e., cytostatic compounds). A preferred compound for inducing cell death is the compound F16, which has been shown to inhibit cell proliferation first and then to induce apoptosis of cells.

Thus, the invention provides methods for treating a subject having an unwanted cell proliferation or differentiation, e.g., a subject having a proliferative disease. The unwanted cell proliferation can be benign or malignant. In a preferred embodiment, the abnormal cell proliferation results from a defect in a protein transmitting a growth signal to the nucleus, such as those named above. Exemplary conditions that are characterized by such an abnormal cell proliferation and which can be treated according to the methods of the invention are further described herein. In one embodiment, the invention provides a method for treating oncogene-mediated, transformation, tumorigenesis or metastasis. In particular, this invention provides methods for the suppression of oncogenesis that is mediated by the HER-2/c-erb B-2/neu oncogene, an oncogene which has been correlated with a poor prognosis of breast and ovarian carcinoma in humans. The invention also relates to the suppression of oncogenesis mediated by Ras. In addition to various forms of cancers, disorders associated with excessive cellular proliferation resulting from, e.g., infection by a pathogen, such as a virus (e.g., Human Papilloma Virus (HPV)) can also be treated according to the methods of the invention.

The compounds of the invention are also useful as markers of certain types of cells, such as cells proliferating excessively, in particular transformed cells. It has been shown herein that cells that are killed by F16 or QR fluoresce before being killed. Thus, F16 and QR can be used not only to kill cells, but also to label cells that will be killed or that are being killed by it.

Also within the scope of the invention are screening assays, such as those described in the Examples, allowing for the isolation of compounds, such as small molecules, inhibiting proliferation of cells that are excessively proliferating, e.g., transformed cells. In one embodiment, the method comprises (i) transfecting into normally proliferating cells a construct containing an oncogene, such that the cells become transformed as a result of the expression of the oncogene product; (ii) contacting the transformed cells and the normal counterpart cells with a compound from a library of compounds, such as small molecules for an amount of time appropriate for affecting their proliferation; and (iii) comparing the proliferation of the transformed cells and that of their normal counterpart in the presence and absence of the compound, such that a reduction in the proliferation of transformed cells relative to that of the normal cells in the presence of the compound indicates that the compound specifically inhibits proliferation of transformed cells.

Definitions

For convenience, the meaning of certain terms and phrases employed in the specification, examples, and appended claims are provided below.

The terms “a” and “an” refer to “one or more” when used in this application, including the claims.

“Abnormal growth of cells” means cell growth independent of normal regulatory mechanisms (e.g., loss of contact inhibition), including abnormal growth resulting form expression of an oncogene. An abnormal growth of cells can be a slower growth or a faster growth.

The term “analog” of a compound refers to a compound having a substantial structural similarity to a particular compound and having essentially the same biological activity as the compound.

An “anti-neoplastic agent” refers to a chemotherapeutic agent effective against cancer.

“c-erbB” refers to a cellular gene which encodes the epidermal growth factor receptor (EGFr). The c-erbB gene is a member of the tyrosine-specific protein kinase family to which many proto-oncogenes belong. The erbB gene is an oncogene form of c-erbB which is present in the avian erythroblastosis virus and which encodes a protein which lacks essentially all of the extracellular domain which renders it constitutively active.

“c-erbB-2”, which is referred to herein interchangeably as “erbB2”, “HER-2”, “Neu”, “Neu^{T}” or “neu oncogene”), encodes a p185 tumor antigen which is a growth factor receptor having extracellular, transmembrane, and intracellular domains. This protein differs from its normal counterpart, the neu proto-oncogene or epidermal growth factor receptor (EGFr) in a single amino acid (point mutation) located in the transmembrane domain, causing the receptor to become constitutively active, i.e., active in the absence of ligand.

The term “compound” refers generally to a molecule. A compound can be an inorganic or organic molecule, e.g., a peptide, protein, nucleic acid. A compound is preferably a small molecule. “Compounds of the invention” include compounds having a general formula A, B, C, D, E, F, G, H, I, J and/or K, further described herein, which modulate cell proliferation and/or differentiation or which induce cell death. Preferred compounds of the invention are any of the following compounds E20 (ID 141525); F12 (ID 120590); F16 (ID 274873); H10 (ID 120670); J6 (ID 120856); N12 (ID 215015); L4 (ID 121113); B15 (ID217496) and QR, as well as derivatives, analogs and prodrugs thereof which modulate cell proliferation and/or differentiation or which induce cell death.

The term “antiproliferative” therapeutic or compound refers to a compound or therapeutic which inhibits cell proliferation.

The term “cytostatic” when referring to the activity of a compound means that the compound causes the cell to cell cycle arrest, but it does not kill the cell. Thus, removal of the drug from the environment of the cell may result in the regain of cell proliferation.

The term “derivative” of a compound or of a small molecule refers to a compound which can be derived, e.g., by chemical synthesis, from the original compound. Thus a derivative of a compound has certain structural similarities with the compound.

The term “unwanted cell proliferation” refers to cell proliferation that is undesirable. Unwanted cell proliferation can refer to cells which are proliferating normally and to cells which are proliferating abnormally, such as cancerous cells. For example, a wart is a tissue in which unwanted epithelial cell proliferation is occurring.

The terms “excessive cell proliferation,” used interchangeably herein with “hyper-proliferation” of cells refers to cells, which divide more often than their normal or wild-type counterpart or which are not sensitive to normal mechanisms of growth control. For example, cells are excessively proliferating when they double in less than 24 hours if their normal counterparts double in 24 hours. Excessive proliferation can be detected by simple counting of the cells, with or without specific dyes, or by detecting DNA replication or transcription, such as by measuring incorporation of a labeled molecule or atom into DNA or RNA.

“Inhibiting cell proliferation” refers to decreasing the rate of cell division, by arresting or slowing down the cell cycle. The term refers to complete blockage of cell proliferation, i.e., cell cycle arrest, as well as to a lengthening of the cell cycle. For example, the period of a cell cycle can be increased by about 10%, about 20%, about 30, 40, 50, or 100%. The duration of the cell cycle can also be augmented by a factor of two, three, 4, 5, 10 or more.

“Normalizing cell proliferation” refers to reducing the rate of cell proliferation of a cell that proliferates excessively relative to that of its normal or wild-type counterpart, or increasing the rate of cell proliferation of a cell that proliferates poorly relative to its normal or wild-type counterpart.

“Modulating cell differentiation” refers to the stimulation or inhibition of cell differentiation.

The term “oncogene” refers to a gene which is associated with certain forms of cancer. Oncogenes can be of viral origin or of cellular origin. An oncogene is a gene encoding a mutated form of a normal protein or is a normal gene which is expressed at an abnormal level, e.g., over-expressed. Over-expression can be caused by a mutation in a transcriptional regulatory element, e.g., the promoter, or by chromosomal rearrangement resulting in subjecting the gene to an unrelated transcriptional regulatory element. The normal cellular counterpart of an oncogene is referred to as “proto-oncogene.” Proto-oncogenes generally encode proteins which are involved in regulating cell growth, and are often growth factor receptors. Numerous different oncogenes have been implicated in tumorigenesis. Tumor suppressor genes, e.g., p53 or p53-like genes are also encompassed by the term “proto-oncogene.” Thus, a mutated tumor suppressor gene which encodes a mutated tumor suppressor protein or which is expressed at an abnormal level, in particular an abnormally low level, is referred to herein as “oncogene.” The terms “oncogene protein” refer to a protein encoded by an oncogene.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention

“Suppression of an oncogenic phenotype” of a cell refers to a reduction in the transforming, tumorigenic or metastatic potential of the cell.

The term “transformed cell” refers to a cell which was converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. Transformed cells include cancer cells, such as cells over-expressing a proto-oncogene or expressing a mutated form of an proto-oncogene (i.e., an oncogene). Transformed cells also include cells infected by a microorganism, e.g., viruses. Exemplary viruses are retroviruses.

The term “proliferative disorder” refers to any disease/disorder of a tissue marked by unwanted or aberrant proliferation of at least some cells in the tissue. Such diseases include cancer, as well as benign diseases or disorders, such as warts or other benign tumors.

Throughout this application, the term “proliferative skin disorder” refers to any disease/disorder of the skin marked by unwanted or aberrant proliferation of cutaneous tissue. These conditions are typically characterized by epidermal cell proliferation or incomplete cell differentiation, and include, for example, X-linked ichthyosis, psoriasis, atopic dermatitis, allergic contact dermatitis, epidermolytic hyperkeratosis, and seborrheic dermatitis. For example, epidermodysplasia is a form of faulty development of the epidermis. Another example is “epidermolysis”, which refers to a loosened state of the epidermis with formation of blebs and bullae either spontaneously or at the site of trauma.

The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction; “squamous cell carcinoma”, which refers to carcinomas arising from squamous epithelium and having cuboid cells; “carcinosarcoma”, which include malignant tumors composed of carcinomatous and sarcomatous tissues; “adenocystic carcinoma”, carcinoma marked by cylinders or bands of hyaline or mucinous stroma separated or surrounded by nests or cords of small epithelial cells, occurring in the mammary and salivary glands, and mucous glands of the respiratory tract; “epidermoid carcinoma”, which refers to cancerous cells which tend to differentiate in the same way as those of the epidermis; i.e., they tend to form prickle cells and undergo cornification; “nasopharyngeal carcinoma”, which refers to a malignant tumor arising in the epithelial lining of the space behind the nose; and “renal cell carcinoma”, which pertains to carcinoma of the renal parenchyma composed of tubular cells in varying arrangements. Another carcinomatous epithelial growth is “papillomas”, which refers to benign tumors derived from epithelium and having a papillomavirus as a causative agent; and “epidermoidomas”, which refers to a cerebral or meningeal tumor formed by inclusion of ectodermal elements at the time of closure of the neural groove.

As used herein, the term “psoriasis” refers to a hyperproliferative skin disorder which alters the skin's regulatory mechanisms. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the dermis layer and polymorphonuclear leukocyte infiltration into the epidermis layer resulting in an increase in the basal cell cycle. Additionally, hyperkeratotic and parakeratotic cells are present.

The term “keratosis” refers to proliferative skin disorder characterized by hyperplasia of the horny layer of the epidermis. Exemplary keratotic disorders include keratosis follicularis, keratosis palmaris et plantaris, keratosis pharyngea, keratosis pilaris, and actinic keratosis.

As used herein, “immortalized cells” refers to cells which have been altered via chemical and/or recombinant means such that the cells have the ability to grow through an indefinite number of divisions in culture.

A “patient” or “subject” can mean either a human or non-human animal , e.g., an ovine, bovine, porcine, equine, bird, canine, feline or non-human primate.

The term “cosmetic preparation” refers to a form of a pharmaceutical preparation which is formulated for topical administration.

An “effective amount” of a compound of the invention, with respect to the subject method of treatment, refers to an amount of a compound of the invention in a preparation which, when applied as part of a desired dosage regimen brings about a change in the rate of cell proliferation and/or the state of differentiation of a cell or cell kiling so as to produce a result according to clinically acceptable standards for the disorder to be treated or the cosmetic purpose.

The “growth state” of a cell refers to the rate of proliferation of the cell and the state of differentiation of the cell.

“Treating” a subject refers to curing, preventing or improving at least one symptom of the disease. For example, treating cancer in a subject includes preventing cancer, reducing tumor load or curing the subject.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (□) constant. This well known constant is described in many references, for instance, J. March, Advanced Organic Chemistry, McGraw Hill Book Company, New York, (1977 edition) pp. 251-259. The Hammett constant values are generally negative for electron donating groups (□[P]=−0.66 for NH₂) and positive for electron withdrawing groups (□[P]=0.78 for a nitro group), □[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, azetidine, azepine, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

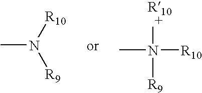

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

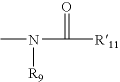

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

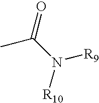

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

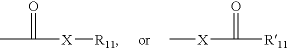

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

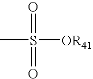

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

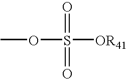

in which R₄₁ is as defined above.

The term “sulfonamido” is art recognized and includes a moiety that can be represented by the general formula:

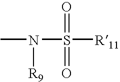

in which R₉ and R′₁₁ are as defined above.

The term “sulfamoyl” is art-recognized and includes a moiety that can be represented by the general formula:

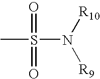

in which R₉ and R₁₀ are as defined above.

The term “sulfonyl”, as used herein, refers to a moiety that can be represented by the general formula:

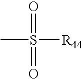

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

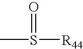

in which R₄₄ is selected from the group consisting of hydrogen, alkyl, alkenyl, alkynyl, cycloalkyl, heterocyclyl, aralkyl, or aryl.

A “phosphoryl” can in general be represented by the formula:

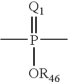

wherein Q₁ represented S or O, and R₄₆ represents hydrogen, a lower alkyl or an aryl. When used to substitute, e.g., an alkyl, the phosphoryl group of the phosphorylalkyl can be represented by the general formula:

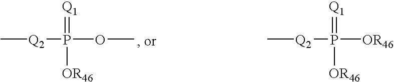

wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, it may be isolated using chiral chromatography methods, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as inhibitors of cell proliferation), wherein one or more simple variations of substituents are made which do not adversely affect the efficacy of the compound inhibiting cell proliferation. In general, the compounds of the present invention may be prepared by methods known in the art, using readily available starting materials, reagents and conventional synthesis procedures. In these reactions, it is also possible to make use of variants which are in themselves known, but are not mentioned here.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

For purposes of this invention, the chemical elements are identified in accordance with the Periodic Table of the Elements, CAS version, Handbook of Chemistry and Physics, 67th Ed., 1986-87, inside cover.

Compounds of the Invention

In certain embodiments, a compound of the present invention is represented by A:

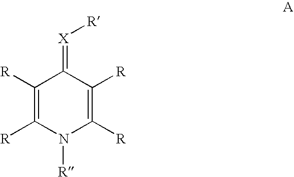

wherein

X represents N or CR;

R represents independently for each occurrence H, halogen, alkyl, alkenyl, alkynyl, hydroxyl, alkoxyl, silyloxy, amino, nitro, sulfhydryl, alkylthio, imine, amide, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ester, heteroalkyl, nitrile, guanidine, amidine, acetal, ketal, amine oxide, aryl, heteroaryl, azide, aziridine, carbamate, epoxide, hydroxamic acid, imide, oxime, sulfonamide, thioamide, thiocarbamate, urea, thiourea, or —(CH₂)ₘ—R₈₀;

R′ represents independently for each occurrence H, alkyl, heteroalkyl, aryl, heteroaryl, aralkyl, heteroaralkyl, alkylC(O)alkyl, arylC(O)alkyl, or —(CH₂)ₘ—R₈₀;

R″ represents independently for each occurrence H, alkyl, heteroalkyl, aryl, heteroaryl, aralkyl, heteroaralkyl, formyl, acyl, 2-aminoacyl, 3-aminoacyl, alkylC(O)alkyl, arylC(O)alkyl, sulfonyl, alkoxycarbonyl, alkylaminocarbonyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure A and the attendant definitions, wherein R represents independently for each occurrence H, alkyl, heteroalkyl, aryl, heteroaryl, aralkyl, heteroaralkyl, or —(CH₂)ₘ—R₈₀.

In certain embodiments, the compounds of the present invention are represented by structure A and the attendant definitions, wherein R represents independently for each occurrence H.

In certain embodiments, a compound of the present invention is represented by B:

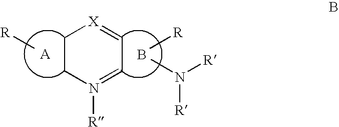

wherein

A and B are substituted or unsubstituted aryls, preferably substituted with one or more hydrophobic groups;

X represents N or CR;

R represents independently for each occurrence H, halogen, alkyl, alkenyl, alkynyl, hydroxyl, alkoxyl, silyloxy, amino, nitro, sulfhydryl, alkylthio, imine, amide, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ester, heteroalkyl, nitrile, guanidine, amidine, acetal, ketal, amine oxide, aryl, heteroaryl, azide, aziridine, carbamate, epoxide, hydroxamic acid, imide, oxime, sulfonamide, thioamide, thiocarbamate, urea, thiourea, or —(CH₂)ₘ—R₈₀;

R′ represents independently for each occurrence H, alkyl, heteroalkyl, aryl, heteroaryl, aralkyl, heteroaralkyl, formyl, acyl, 2-aminoacyl, 3-aminoacyl, alkylC(O)alkyl, arylC(O)alkyl, sulfonyl, alkoxycarbonyl, alkylaminocarbonyl, or —(CH₂)ₘ—R₈₀;

R″ represents independently for each occurrence H, alkyl, heteroalkyl, aryl, heteroaryl, aralkyl, heteroaralkyl, alkylC(O)alkyl, arylC(O)alkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure B and the attendant definitions, wherein X represents independently for each occurrence N.

In certain embodiments, the compounds of the present invention are represented by structure B and the attendant definitions, wherein R represents independently for each occurrence H, alkyl, heteroalkyl, aryl, heteroaryl, aralkyl, heteroaralkyl, or —(CH₂)ₘ—R₈₀.

In certain embodiments, the compounds of the present invention are represented by structure B and the attendant definitions, wherein R represents independently for each occurrence H.

In certain embodiments, the compounds of the present invention are represented by structure B and the attendant definitions, wherein R″ represents independently for each occurrence alkyl.

In certain embodiments, a compound of the present invention is represented by C:

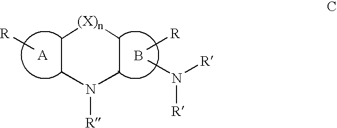

wherein

A and B are substituted or unsubstituted aryls, preferably substituted with one or more hydrophobic groups;

X represents C(R)₂;

R represents independently for each occurrence H, halogen, alkyl, alkenyl, alkynyl, hydroxyl, alkoxyl, silyloxy, amino, nitro, sulfhydryl, alkylthio, imine, amide, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ester, heteroalkyl, nitrile, guanidine, amidine, acetal, ketal, amine oxide, aryl, heteroaryl, azide, aziridine, carbamate, epoxide, hydroxamic acid, imide, oxime, sulfonamide, thioamide, thiocarbamate, urea, thiourea, or —(CH₂)ₘ—R₈₀;

R′ represents independently for each occurrence H, alkyl, heteroalkyl, aryl, heteroaryl, aralkyl, heteroaralkyl, formyl, acyl, 2-aminoacyl, 3-aminoacyl, alkylC(O)alkyl, arylC(O)alkyl, sulfonyl, alkoxycarbonyl, alkylaminocarbonyl, or —(CH₂)ₘ—R₈₀;

R″ represents independently for each occurrence H, alkyl, heteroalkyl, aryl, heteroaryl, aralkyl, heteroaralkyl, formyl, acyl, 2-aminoacyl, 3-aminoacyl, alkylC(O)alkyl, arylC(O)alkyl, sulfonyl, alkoxycarbonyl, alkylaminocarbonyl, or —(CH₂)ₘ—R₈₀;

R₈₀ represents independently for each occurrence aryl, cycloalkyl, cycloalkenyl, heterocyclyl, or polycyclyl;

m is an integer in the range 0 to 8 inclusive; and

n represents an integer selected from the group consisting of 0, 1, 2, and 3.

In certain embodiments, the compounds of the present invention are represented by structure C and the attendant definitions, wherein X represents independently for each occurrence CH₂.

In certain embodiments, the compounds of the present invention are represented by structure C and the attendant definitions, wherein R represents independently for each occurrence H, alkyl, heteroalkyl, aryl, heteroaryl, aralkyl, heteroaralkyl, or —(CH₂)ₘ—R₈₀.

In certain embodiments, the compounds of the present invention are represented by structure C and the attendant definitions, wherein R represents independently for each occurrence H.

In certain embodiments, the compounds of the present invention are represented by structure C and the attendant definitions, wherein n represents 1 or 2.

In certain embodiments, a compound of the present invention is represented by D:

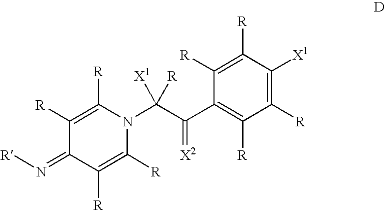

wherein

X¹ is selected independently for each occurrence from the group consisting of F, Cl, Br, and I and a substituted or unsubstituted benzene ring;

X² is selected from the group consisting of O or S.

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure D and the attendant definitions, wherein X represents Cl or I.

In certain embodiments, the compounds of the present invention are represented by structure D and the attendant definitions, wherein R represents independently for each occurrence H.

In certain embodiments, the compounds of the present invention are represented by structure D and the attendant definitions, wherein R′ represents independently for each occurrence H.

In certain embodiments, the compounds of the present invention are represented by structure D and the attendant definitions, wherein X represents Cl or I; R represents independently for each occurrence H; and R′ represents independently for each occurrence H.

In certain embodiments, a compound of the present invention is represented by E:

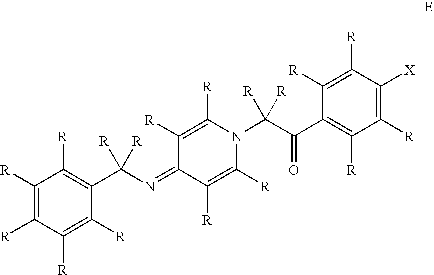

wherein

X is selected from the group consisting of F, Cl, Br, and I;

R represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure E and the attendant definitions, wherein X represents Cl or I.

In certain embodiments, the compounds of the present invention are represented by structure E and the attendant definitions, wherein R represents independently for each occurrence H.

In certain embodiments, the compounds of the present invention are represented by structure E and the attendant definitions, wherein X represents Cl or I; and R represents independently for each occurrence H.

In certain embodiments, a compound of the present invention is represented by F:

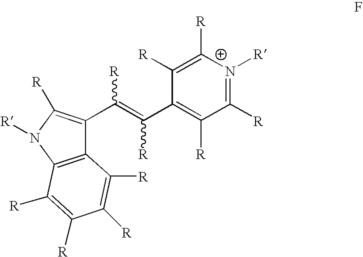

wherein

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure F and the attendant definitions, wherein R represents independently for each occurrence H.

In certain embodiments, the compounds of the present invention are represented by structure F and the attendant definitions, wherein R′ represents independently for each occurrence alkyl.

In certain embodiments, the compounds of the present invention are represented by structure F and the attendant definitions, wherein R represents independently for each occurrence H; and R′ represents independently for each occurrence alkyl.

In certain embodiments, a compound of the present invention is represented by G:

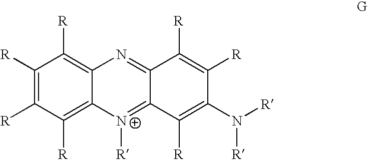

wherein

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure G and the attendant definitions, wherein R represents independently for each occurrence H.

In certain embodiments, the compounds of the present invention are represented by structure G and the attendant definitions, wherein R′ represents independently for each occurrence alkyl.

In certain embodiments, the compounds of the present invention are represented by structure G and the attendant definitions, wherein R represents independently for each occurrence H; and R′ represents independently for each occurrence alkyl.

In certain embodiments, a compound of the present invention is represented by H:

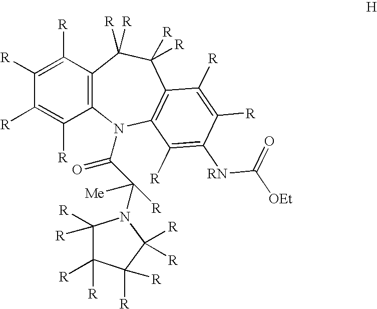

wherein

R represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure H and the attendant definitions, wherein R represents independently for each occurrence H.

In certain embodiments, a compound of the present invention is represented by I:

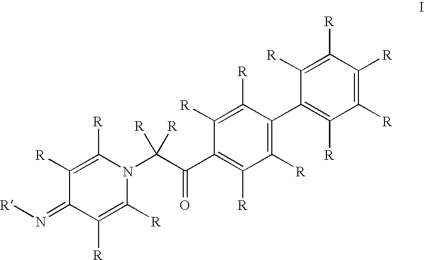

wherein

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure I and the attendant definitions, wherein R represents independently for each occurrence H.

In certain embodiments, the compounds of the present invention are represented by structure I and the attendant definitions, wherein R′ represents independently for each occurrence H.

In certain embodiments, the compounds of the present invention are represented by structure I and the attendant definitions, wherein R represents independently for each occurrence H; and R′ represents independently for each occurrence H.

In certain embodiments, a compound of the present invention is represented by J:

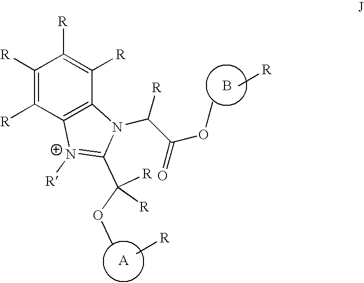

wherein

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure J and the attendant definitions, wherein R represents independently for each occurrence H.

In certain embodiments, the compounds of the present invention are represented by structure J and the attendant definitions, wherein R′ represents independently for each occurrence H.

In certain embodiments, the compounds of the present invention are represented by structure J and the attendant definitions, wherein R represents independently for each occurrence H; and R′ represents independently for each occurrence H.

In certain embodiments, a compound of the present invention is represented by K:

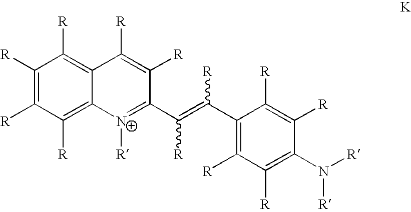

wherein

R represents independently for each occurrence H, alkyl, heteroalkyl, aryl, heteroaryl, aralkyl, heteroaralkyl, or —(CH₂)ₘ—R₈₀;

R′ represents independently for each occurrence H, alkyl, heteroalkyl, aryl, heteroaryl, aralkyl, heteroaralkyl, or —(CH₂)ₘ—R₈₀;

R₈₀ represents independently for each occurrence aryl, cycloalkyl, cycloalkenyl, heterocyclyl, or polycyclyl; and

m is an integer in the range 0 to 8 inclusive.

In certain embodiments, the compounds of the present invention are represented by structure K and the attendant definitions, wherein R represents independently for each occurrence H.

In certain embodiments, the compounds of the present invention are represented by structure K and the attendant definitions, wherein R′ represents independently for each occurrence alkyl.

In certain embodiments, the compounds of the present invention are represented by structure K and the attendant definitions, wherein R represents independently for each occurrence H; and R′ represents independently for each occurrence alkyl.

In certain embodiments, the compounds of the present invention are represented by structure K and the attendant definitions, wherein R represents independently for each occurrence H; and R′ represents independently for each occurrence methyl or ethyl.

The above-described compounds can be prepared according to methods known in the art, e.g., as described in “Comprehensive Organic Transformations: A Guide to Functional Group Preparations” by Richard C. Larock, VCH Publishers (1989) and “Advanced Organic Chemistry: Reactions, Mechanisms, and Structure” Fourth Edition, Jerry March, Wiley Interscience (1992). At least some of the steps of the synthesis can be identical to those used to prepare the chemical library from which some of the compounds of the invention were isolated. In addition, the compounds of the invention can be purchased from companies, e.g., companies involved in custom synthesis of chemical compounds. Exemplary companies which synthesize compounds, e.g., F16, are Interbioscreen Ltd., Moscow, Russia (the catalogue number for F16 is 2S-13965); Asinex Ltd., Moscow, Russia (the catalogue number for F16 is BAS 1027104); and ChemDiv, Inc., U.S. QR is available from Aldrich-Sigma, under catalog no. 20,131-6.

Assays for Testing the Activity of Compounds

The activity of compounds can be tested by incubation with cells and determining the effect on the proliferation of the cells or their effect on the survival of the cells, as described in the Examples. Accordingly, the effect of the compound can be tested by incubating cells with varying amounts of the compounds and determining the amount of DNA at various times after the addition of the compound to the cells. Cell proliferation can also be determined by counting the cells at different times. Viable cells can be counted by staining the cells with a specific dye, e.g., Trypan Blue, according to methods well known in the art. Other methods include measuring the incorporation of a labeled molecule into DNA or RNA or protein of cells. For example, cell proliferation is often by measured by ³H thymidine or 5-bromodeoxyuridine incorporation assays, also well known in the art.

Methods for identifying the extent of cell killing, such as by apoptosis are also well known in the art and described in the Examples. For example, cells having undergone apoptosis have certain morphologic characteristics and DNA that is fragmented and takes the appearance of a ladder of DNA fragments in a gel electrophoresis experiment.

The effect of a compound on cell differentiation can be determined by visualization of the cells after having been contacted with the compound, preferably by comparison with cells which have not been contacted with the compound. The differentiation of certain cells is visible by the naked eye (e.g., that of 3T3L1 cells), whereas that of other cells may require the use of a microscope. Specific dyes can also be used to evaluate the state of differentiation of cells. Cell differentiation can also be monitored by measuring the expression level of certain genes, whose expression is known to vary during differentiation of the cells.

Cells that can be used for testing compounds of the invention include cell lines and primary cell cultures. Numerous cell lines that are transformed by over-expression of a proto-oncogene or the presence of an oncogene are available, e.g., from the American Type Culture Collection (ATCC). Primary cell cultures can be established from biopsies obtained from subjects. For example, primary tissue cultures of cells over-expressing an activated form of Neu can be prepared from biopsies of subjects having breast cancer. Cell lines over-expressing a gene, e.g., a proto-oncogene can be prepared by transient, or preferably, stable transfection of cells with an expression plasmid containing the gene. Transfection methods are well known in the art and are also described in the examples. Nucleic acids for transforming cells, e.g., proto-oncogenes are also available in the art. Cell lines can also be obtained from transgenic animals expressing an oncogene. For example, MG 1361 is a breast carcinoma cell line obtained from the MMTV-neu transgenic mouse (Sacco et al., Breast Cancer Res. Treat., 47:171-180 (1998)).

The present invention also provides methods of testing a compound (e.g., the candidate drug) for its antiproliferative effect; its effect on cell differentiation; its induction of cell death; and/or its toxicity on normal or wild-type cells in a transgenic animal, e.g., mouse. Transgenic mice can be produced that express a transforming agent (e.g., a growth factor receptor) under the control of a promoter, e.g., a tissue specific promoter. Such mice develop carcinomas that have genetic and pathological features that closely resemble human cancers. For example, in a MMTV-neu transgenic mouse lineage, 100% of the female mice develop mammary adenocarcinomas (Sacco et al., Gene Therapy 2:493497 (1995); Sacco et al., Gene Therapy 5:383-393 (1998)). The ability of the compound to inhibit tumor formation or growth can then be ascertained. In one embodiment, the effect on tumors is monitored by determining the tumor size and/or weight. The compounds can be administered by a variety of ways including orally, subcutaneously, or intraperitoneally. Generally, at least two groups of animals are used in the assay, with at least one group being a control group, e.g., receiving the vehicle without the compound.

Other animals transgenic for an oncogene are described in the following publications. MMTV-neu transgenic mice are described in Muller et al. (1988) Cell 54:105. A neu transgenic mouse in which the Neu coding region is under the control of the normal neu promoter is described in Weinstein et al. (2000) Mol. Med. 6:4. A transgenic mouse expressing activated ras is described, e.g., in Kohl, et al., Nature Medicine, vol. 1, No. 8 (August 1995). A myc transgenic mouse is described in U.S. Pat. No. 5,925,803, by Leder et al., which also describes methods for making mice that are transgenic for any one of a variety of oncogenes. Myc and ras transgenic animals are further described in Muller et al. (1988) Cell 54:105; Pattengale et al. (1989) Am J. Pathol. 135:39; and Cardiff et al. (1991) Am J. Path. 139:495. A transgenic mouse, transgenic for the SV40 TAg transgene, susceptible to developing prostate cancer is described in U.S. Pat. No. 5,917,124. Yet other trangenic animals for testing derivatives and analogs of the compounds identified and described herein are described in the art.

Other cells proliferating in an unwanted manner, e.g., abnormally, can be obtained from the American Type Culture Collection (ATCC), and other animal models in which certain cells proliferate or differentiate in an undesirable manner are known in the art.

Diseases and Disorders that can be Treated with the Compounds of the Invention

Generally, the compounds of the invention can be used to normalize, e.g., inhibit or block the proliferation of cells and/or modulate the differentiation of cells. The compounds can also be used to kill target cells, e.g., by inducing apoptosis in the cells. The cells whose proliferation or differentiation is to be normalized or which are to be killed are referred herein as “target cells.” A target cell can essentially be any cell whose proliferation is to be inhibited or its differentiation modulated or which is to be killed. For example, target cells can be cells which are defective, e.g., non-responsive to, normal cell proliferation control mechanisms. In one embodiment, target cells are transformed cells, e.g., cells which express a mutated form of a molecule, e.g., a protein, or which over-express a molecule, e.g., a protein. As further discussed herein, a target cell can be a cell which is defective in its response to a growth factor, e.g., EGF. Thus, preferred target cells are those containing a defect in a growth factor receptor or signal transduction molecule relaying the information to the nucleus. Exemplary target cells are those which express an activated form of a proto-oncogene, e.g., the Neu oncogene or the Ras oncogene.

Accordingly, a target cell can be not only a cell expressing the Neu or Ras or Myc oncogene or β-catenin (as described in the Examples), but it can also be a cell expressing another oncogene. Indeed, in view of the interconnection between different signal transduction pathways, a compound which has an antiproliferative effect against a cell transformed with a Neu oncogene can also have an antiproliferative effect against a cell having another defect, e.g., the presence of an oncogene. In other terms, if a cell has a defect in a particular molecule of a signal transduction pathway, the cell can be “cured” not only by effecting that particular molecule, but by compensating for the effect of the defect in the particular molecule by effecting upstream or downstream molecules in the same signal transduction pathway, or in a parallel signal transduction pathway. At least for this reason, the compounds of the invention are expected to be effective against a broad range of target cells, and not only target cells transformed with an oncogene. In addition, at least some of the compounds of the invention may be effective against cells which proliferate and/or differentiate normally, i.e., wild-type cells. For example, certain compounds could be used to arrest cell proliferation.

Preferred compounds of the invention are those which act specifically, or essentially specifically, on the target cell. As shown herein, the compounds of the invention were shown to inhibit cell proliferation of the target transformed epithelial cells EpH4, without significantly affecting proliferation of the counterpart non-transformed cells.

Although the EpH4 cells against which the compounds of the invention were shown to be effective (see Examples) are epithelial cells, the compounds of the invention would also be effective against other cell types, since signal transduction pathways are shared by all cells. Accordingly, target cells of the invention can be epithelial cells, but also fibroblasts, neural cells, muscle cells, lymphocytes, macrophages, or any other cell type. They can be differentiated cells or precursor cells, e.g., embryonic stem cells.

In addition, the target cells can be mammalian cells, e.g., human, canine, feline, bovine, ovine, murine, and rat. Non-mammalian cells, which share essentially the same signal transduction pathways as those in mammalian cells, e.g., yeast cells, can also be target cells of the invention.

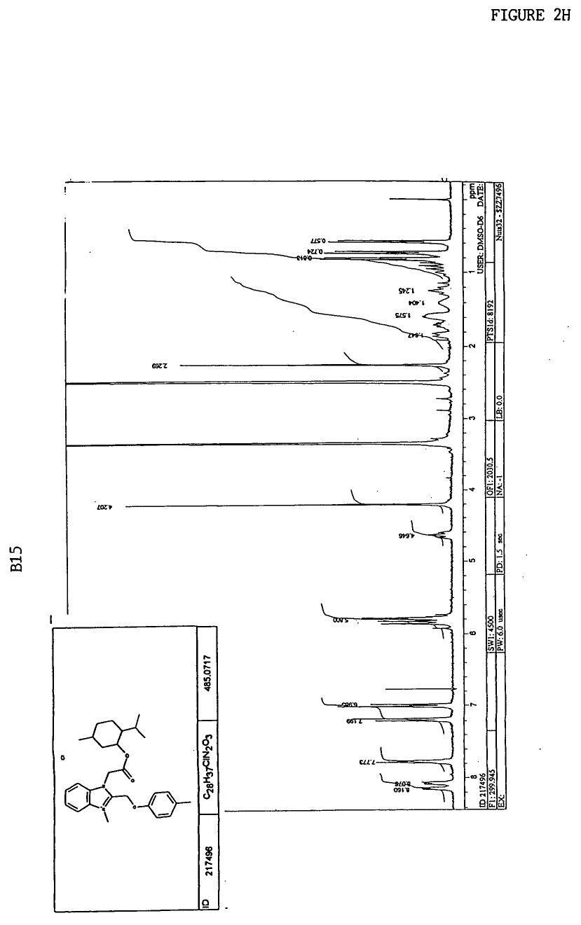

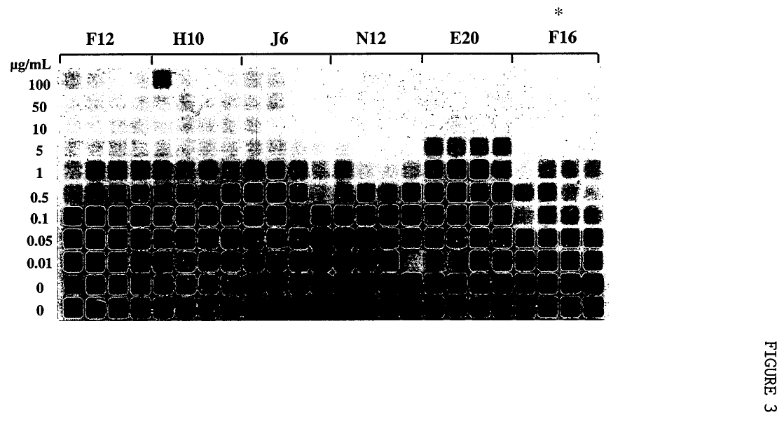

The efficacy of the compounds of the invention against a broad range of target cells allows for broad applications for these compounds. The following are exemplary therapeutic applications for the compounds of the invention. In regards to therapeutic applications, it must be pointed out that the compounds of the invention are effective at very low doses. For example, F16 is effective at 10 nM (see, e.g., FIG. 3). In addition, these compounds were shown not to affect certain other cells. For example, most compounds did not significantly inhibit proliferation of the counter-part non-transformed cells and some of the compounds only affected cells transformed with certain oncogenes and not others.

In one embodiment, the invention provides methods for treating cancer, e.g., cancers that are caused by, or associated with, expression of an oncogene or over-expression of a proto-oncogene, e.g., the Neu proto-oncogene. An exemplary cancer that can be treated is breast cancer, in particular, forms of breast cancers which are associated with an overexpression of the Neu proto-oncogene.

Amplification and/or overexpression of the human erbB2 gene correlates with a poor prognosis in breast and ovarian cancers, in particular, carcinomas. Slamon et al., Science 235:177-82 (1987); Slamon et al., Science 244:707-12 (1989). Overexpression of erbB2 has been correlated with other carcinomas including carcinomas of the stomach, endometrium, salivary gland, lung, kidney, colon and bladder. Accordingly, in U.S. Pat. No. 4,968,603, Slamon et al. describe and claim various diagnostic assays for determining erbB2 gene amplification or expression in tumor cells. Slamon et al. discovered that the presence of multiple copies of the erbB2 oncogene in tumor cells indicates that the disease is more likely to spread beyond the primary tumor site, and that the disease may therefore require more aggressive treatment than might otherwise be indicated by other diagnostic factors. Slamon et al. conclude that the erbB2 gene amplification test, together with the determination of lymph node status, provides greatly improved prognostic utility.

Although the compounds of the invention have been isolated based on their ability to inhibit growth of cells that are transformed with an activated form of the Neu proto-oncogene, in view of the similarity of the Neu proto-oncogene with other members of the family of EGF receptors, the compounds of the invention are likely to similarly inhibit the proliferation of cells transformed with other members of the EGF receptor family, such as Neu-erb2-related genes. Accordingly, the compounds of the invention can be used for treating diseases, in particular, malignancies, that are associated with the presence of such related genes. The effectiveness of the compounds in treating these other diseases can be tested using appropriate cell lines or animal models, as further described herein.

For example, the Neu proto-oncogene is related to erbB1, a 170 kDa protein, which has been causally implicated in human malignancy. In particular, increased expression of this gene has been observed in more aggressive carcinomas of the breast, bladder, lung, and stomach. erbB gene amplification or overexpression, or a combination of both, has been demonstrated in squamous cell carcinomas and glioblastomas (Libermann, T. A., Nusbaum, H. R., Razon, N., Kris, R., Lax, I., Soreq, H., Whittle, N., Waterfield, M. D., Ullrich, A. & Schlessinger, J., 1985, Nature 313:144-147). Accordingly, the compounds of the invention are believed to be useful for treating these malignancies.

A further related gene of the Neu proto-oncogene is erbB3 (or HER3), which encodes the ErbB-3 receptor (p180.sup.HER3). This receptor has been described, e.g., in U.S. Pat. Nos. 5,183,884 and 5,480,968; Kraus et al., PNAS USA 86:9193-97 (1989); EP Patent Application No. 444,961A1; Kraus et al., PNAS USA 90:2900-04 (1993). Kraus et al. (1989) discovered that markedly elevated levels of erbB3 mRNA were present in certain human mammary tumor cell lines indicating that erbB3, like erbB1 and erbB2, may play a role in human malignancies. Also, Kraus et al. (1993) showed that EGF-dependent activation of the ErbB-3 catalytic domain of a chimeric EGFR/ErbB-3 receptor resulted in a proliferative response in transfected NIH-3T3 cells. Furthermore, these researchers demonstrated that some human mammary tumor cell lines display a significant elevation of steady-state ErbB-3 receptor tyrosine phosphorylation, further implicating this receptor in human malignancies. The role of erbB3 in cancer has been explored by others, and this gene has been found to be overexpressed in breast (Lemoine et al., Br. J. Cancer 66:1116-21 [1992]), gastrointestinal (Poller et al., J. Pathol. 168:275-80 [1992]; Rajkumer et al., J. Pathol. 170:271-78 [1993]; Sanidas et al., Int. J. Cancer 54:935-40 [1993]), and pancreatic cancers (Lemoine et al., J. Pathol. 168:269-73 [1992], and Friess et al., Clinical Cancer Research 1:1413-20 [1995]). Yet another member of the class I subfamily of growth factor receptor protein tyrosine kinases (EGF receptor family) has been further extended to include the ErbB-4 (HER4) receptor, which is the product of the erbB4 (HER4) gene. See EP Patent Application No. 599,274; Plowman et al., PNAS USA 90:1746-50 (1993); and Plowman et al., Nature 366:473-75 (1993). Plowman et al. found that increased erbB4 expression closely correlated with certain carcinomas of epithelial origin, including breast adenocarcinomas. Diagnostic methods for detection of human neoplastic conditions (especially breast cancers) that evaluate erbB4 expression are described in EP Patent Application No. 599,274.

The invention further provides methods for treating proliferative diseases which are associated with the presence of an activated form of the Ras proto-oncogene. As described in the Examples, certain compounds that were identified as having an antiproliferative activity against cells expressing an activated form of the Neu proto-oncogene are also effective against cells expressing an activated form of the Ras proto-oncogene.

The Ras protein is part of a signaling pathway that links cell surface growth factor receptors to nuclear signals initiating cellular proliferation. Biological and biochemical studies of Ras action indicate that Ras functions like a G-regulatory protein. In the inactive state, Ras is bound to GDP. Upon growth factor receptor activation, Ras is induced to exchange GDP for GTP and undergoes a conformational change. The GTP-bound form of Ras propagates the growth stimulatory signal until the signal is terminated by the intrinsic GTPase activity of Ras, which returns the protein to its inactive GDP bound form (D. R. Lowy and D. M. Willumsen, Ann. Rev. Biochem. 62:851-891 (1993)). Activation of Ras leads to activation of multiple intracellular signal transduction pathways, including the MAP Kinase pathway and the Rho/Rac pathway (Joneson et al., Science 271:810-812).

Mammalian cells express four types of Ras proteins (H-, N-, K4A-, and K4B-Ras) among which K4B-Ras is the most frequently mutated form of Ras in human cancers. The genes that encode these proteins are abbreviated H-ras, N-ras, K4A-ras and K4B-ras respectively. H-ras is an abbreviation for Harvey-ras. K4A-ras and K4B-ras are abbreviations for the Kirsten splice variants of ras that contain the 4A and 4B exons, respectively.

Mutated ras genes are found in many human cancers, including colorectal carcinoma, exocrine pancreatic carcinoma, and myeloid leukemias. The protein products of these genes are defective in their GTPase activity and constitutively transmit a growth stimulatory signal. Cancers associated with activated Ras that can be treated include epithelial cancers, e.g., prostate cancer, lung cancer, breast cancer, colorectal cancer, and pancreatic cancer.

Activated Myc oncogenes have been shown to cause testicular, breast, lymphocytic (T and B cell) cancers, e.g., pre-B cell lymphomas, Burtkitt's lymphoma as well as cancers of mast cell origin (Leder et al. (1986) Cell 45: 485; Suda et al. (1987) EMBO J. 20: 4055; and Chung et al. (1986) PNAS 83:7918). Accordingly, the compounds of the invention can be used for treating such diseases.

Examples of other proliferative diseases which may be treated with the compounds of the invention include: the benign proliferative disorder neurofibromatosis, or tumors in which ras is activated due to mutation or overexpression of tyrosine kinase oncogenes (e.g., neu, src, abl, lck, lyn, fyn).

Thus, generally, a preferred therapeutic effect provided by the instant composition is the treatment of cancer and specifically the inhibition of cancerous tumor growth and/or the regression of cancerous tumors. The cancer can result from the presence of an oncogene or from another cause. Cancers, which are treatable in accordance with the invention described herein include cancers of the brain, breast, colon, genitourinary tract, prostate, skin, lymphatic system, pancreas (e.g., pancreatic carcinoma such as, for example, exocrine pancreatic carcinoma), rectum, stomach, larynx, ovary, bladder, liver and lung. More particularly, such cancers include histiocytic lymphoma, lung adenocarcinoma, pancreatic carcinoma, colo-rectal carcinoma, small cell lung cancers, bladder cancers, head and neck cancers, acute and chronic leukemias, melanomas, neurological tumor, myeloid leukemias (for example, acute myelogenous leukemia), sarcomas, thyroid follicular cancer, and myelodysplastic syndrome.

Other types of proliferative disorders that can be treated according to the invention include non malignant cell proliferative disorders, e.g., neurofibromatosis, which is a benign proliferative disorder; glaucoma, psoriasis, rheumatoid arthritis, restenosis, inflammatory bowel disease, and chemotherapy-induced alopecia and mucositis. It is believed that such diseases are likely to be sensitive to the compounds of the invention, since the defective cells may have a similar defect in the growth control mechanisms of cells as those which are hyper-proliferative due to the expresssion of an activated form of a proto-oncogene. Furthermore, most of these disorders are related to an abnormal production of, or response to a growth factor, e.g., platelet derived growth factor (PDGF), fibroblast derived growth factor (FGF), epidermal derived growth factor (EGF) and vascular endothelial growth factor (VEGF). Since these factors and their receptors are encoded by genes which, when mutated frequently become oncogenes, the compounds of the invention which are effective to curb proliferation of oncogene transformed cells, are likely to be effective against these other non-cancer proliferative disorders. As further described herein, various in vitro assays and animal models can be used for confirming the likelihood of effect of the compounds in these situations.

The compounds of the invention can also be used for treating smooth muscle cell hyper-proliferation, at least in part since PDGF is considered to be a principal growth-regulatory molecule responsible for smooth muscle cell proliferation. One smooth muscle disorder is atherosclerosis, which is a disease characterized by focal thickening of the inner portion of the artery wall, predisposing an individual to myocardial infarction (heart attack), cerebral infarction (stroke), hypertension (high blood pressure) and gangrene of the extremities. In addition to consisting primarily of proliferated smooth muscle cells, lesions of atherosclerosis are surrounded by large amounts of lipid-laden macrophages, varying numbers of lymphocytes and large amounts of connective tissue. PDGF has been found in numerous cells in such lesions, and it is believed that PDGF plays a critical role in the atherosclerosis disease process. Other smooth muscle diseases include diabetic vascular pathologies.

Another smooth muscle cell disease that can be treated according to the invention is restenosis. This disease is characterized by the regrowth of smooth muscle cells into the lumen of blood vessels following angioplasty or other arterial damage, is a frequent and recurring problem in the long term success of angioplasty. The failure rates of angioplasty as a result of restenosis within six months are reported to be between 25-50% (Leimgruber et al., 1986; Gruentzieg et al., 1987; Nobuyoshi et al., 1988; Serruys et al., 1988). Restenosis also occurs after arterial reconstructions, atherectomy, stent implantation, and laser angioplasty. Injury to arteries during angioplasty results in the activation of medial smooth muscle cells, which begin to migrate and proliferate into the lumen of the artery to form a neointima, or a new layer of cells. It is believed that expansion of this neointima as a result of the new layer of smooth muscle cells, extracellular matrix, and recruited inflammatory cells, is the cause of the eventual reduction of blood flow through the artery and recurrence of ischemic symptoms. The composition of the instant invention is also useful in the prevention of restenosis after percutaneous transluminal coronary angioplasty by inhibiting neointimal formation (C. Indolfi et al. Nature medicine, 1:541-545(1995)).

In another embodiment, the compounds of the invention are used for treating rheumatoid arthritis (R.A.). Synovial tissues of RA patients express high levels of FGF and PDGF compared with synovial tissues of osteoarthritis patients, a non invasive joint disease (Sano et al., J. Cell. Biol. 110:1417-1426, 1990). These data are consistent with the theory that PDGF and FGF play a role in generating an invasive tumor-like behavior in arthritic joints of RA synovial connective tissues (Sano et al., J. Clin. Invest. 91:553-565 (1993)).

Both FGF and VEGF are potent angiogenic factors which induce formation of new capillary blood vessels. Accordingly, the compounds of the invention may be useful in inhibiting vascularization, e.g., in tumors.

The instant composition may also be useful in the treatment and prevention of polycystic kidney disease (D. L. Schaffner et al. American Journal of Pathology, 142:1051-1060 (1993) and B. Cowley, Jr. et al. FASEB Journal, 2:A3160 (1988)).

In addition, the instant compounds may also be useful in the treatment of certain viral infections, in particular in the treatment of hepatitis C or delta and related viruses (J. S. Glenn et al. Science, 256:1331-1333 (1992)). Numerous viruses also induce non cancerous cell proliferation. Examples include papilloma viruses (HPV), which create skin lesions. Such viral infections may also be treatable with the compositions of the invention.

In another aspect of the invention, antiproliferative therapeutics (compounds of the invention) can be used to induce differentiation of epithelially-derived tissue. Such forms of these molecules can provide a basis for differentiation therapy for the treatment of hyperplastic and/or neoplastic conditions involving epithelial tissue. For example, such preparations can be used for the treatment of cutaneous diseases in which there is abnormal proliferation or growth of cells of the skin.

For instance, the pharmaceutical preparations of the invention are intended for the treatment of hyperplastic epidermal conditions, such as keratosis, as well as for the treatment of neoplastic epidermal conditions such as those characterized by a high proliferation rate for various skin cancers, as for example basal cell carcinoma or squamous cell carcinoma. The subject method can also be used in the treatment of autoimmune diseases affecting the skin, in particular, of dermatological diseases involving morbid proliferation and/or keratinization of the epidermis, as for example, caused by psoriasis or atopic dermatosis.

Many common diseases of the skin, such as psoriasis, squamous cell carcinoma, keratoacanthoma and actinic keratosis are characterized by localized abnormal proliferation and growth. For example, in psoriasis, which is characterized by scaly, red, elevated plaques on the skin, the keratinocytes are known to proliferate much more rapidly than normal and to differentiate less completely.

In one embodiment, the preparations of the present invention are suitable for the treatment of dermatological ailments linked to keratinization disorders causing abnormal proliferation of skin cells, which disorders may be marked by either inflammatory or non-inflammatory components. To illustrate, therapeutic preparations of a compound of the invention, e.g., which promotes quiescense or differentiation, can be used to treat varying forms of psoriasis, be they cutaneous, mucosal or ungual. Psoriasis, as described above, is typically characterized by epidermal keratinocytes which display marked proliferative activation and differentiation along a “regenerative” pathway. Treatment with an antiproliferative embodiment of the subject method can be used to reverse the pathological epidermal activition and can provide a basis for sustained remission of the disease.

A variety of other keratotic lesions are also candidates for treatment with the subject antiproliferative preparations. Actinic keratoses, for example, are superficial inflammatory premalignant tumors arising on sun-exposed and irradiated skin. The lesions are erythematous to brown with variable scaling. Current therapies include excisional and cryosurgery. These treatments are painful, however, and often produce cosmetically unacceptable scarring. Accordingly, treatment of keratosis, such as actinic keratosis, can include application, preferably topical, of a composition comprising one or more compounds of the invention in amounts sufficient to inhibit hyperproliferation of epidermal/epidermoid cells of the lesion.

Acne represents yet another dermatologic ailment which may be treated with an antiproliferative embodiment of the subject method. Acne vulgaris, for instance, is a multifactorial disease most commonly occurring in teenagers and young adults, and is characterized by the appearance of inflammatory and noninflammatory lesions on the face and upper trunk. The basic defect which gives rise to acne vulgaris is hypercornification of the duct of a hyperactive sebaceous gland. Hypercornification blocks the normal mobility of skin and follicle microorganisms, and in so doing, stimulates the release of lipases by Propinobacterium acnes and Staphylococcus epidermidis bacteria and Pitrosporum ovale, a yeast. Treatment with an antiproliferative therapeutic, particularly topical preparations, may be useful for preventing the transitional features of the ducts, e.g. hypercornification, which lead to lesion formation. The subject treatment may further include, for example, antibiotics, retinoids and antiandrogens.

The present invention also provides a method for treating various forms of dermatitis. Dermatitis is a descriptive term referring to poorly demarcated lesions which are either pruritic, erythematous, scaley, blistered, weeping, fissured or crusted. These lesions arise from any of a wide variety of causes. The most common types of dermatitis are atopic, contact and diaper dermatitis. For instance, seborrheic dermatitis is a chronic, usually pruritic, dermatitis with erythema, dry, moist, or greasy scaling, and yellow crusted patches on various areas, especially the scalp, with exfoliation of an excessive amount of dry scales stasis dermatitis, an often chronic, usually eczematous dermatitis. Actinic dermatitis is dermatitis that due to exposure to actinic radiation such as that from the sun, ultraviolet waves or x- or gamma-radiation. According to the present invention, the subject therapeutic preparations can be used in the treatment and/or prevention of certain symptoms of dermatitis caused by unwanted proliferation of epithelial cells. Such therapies for these various forms of dermatitis can also include topical and systemic corticosteroids, antipuritics, and antibiotics.

In other embodiments, antiproliferative preparations of therapeutics can be used to inhibit lens epithelial cell proliferation to prevent post-operative complications of extracapsular cataract extraction. Cataract is an intractable eye disease and various studies on a treatment of cataract have been made. But at present, the treatment of cataract is attained by surgical operations. Cataract surgery has been applied for a long time and various operative methods have been examined. Extracapsular lens extraction has become the method of choice for removing cataracts. The major medical advantages of this technique over intracapsular extraction are lower incidence of aphakic cystoid macular edema and retinal detachment. Extracapsular extraction is also required for implantation of posterior chamber type intraocular lenses which are now considered to be the lenses of choice in most cases.

However, a disadvantage of extracapsular cataract extraction is the high incidence of posterior lens capsule opacification, often called after-cataract, which can occur in up to 50% of cases within three years after surgery. After-cataract is caused by proliferation of equatorial and anterior capsule lens epithelial cells which remain after extracapsular lens extraction. These cells proliferate to cause Sommerling rings, and along with fibroblasts which also deposit and occur on the posterior capsule, cause opacification of the posterior capsule, which interferes with vision. Prevention of after-cataract would be preferable to treatment. To inhibit secondary cataract formation, the subject method provides a means for inhibiting proliferation of the remaining lens epithelial cells. For example, such cells can be induced to remain quiescent by instilling a solution containing an antiproliferative therapeutic preparation into the anterior chamber of the eye after lens removal. Furthermore, the solution can be osmotically balanced to provide minimal effective dosage when instilled into the anterior chamber of the eye, thereby inhibiting subcapsular epithelial growth with some specificity.

The subject method can also be used in the treatment of corneopathies marked by corneal epithelial cell proliferation, as for example in ocular epithelial disorders such as epithelial downgrowth or squamous cell carcinomas of the ocular surface.

In another aspect of the invention, the subject method can be used in conjunction with various periodontal procedures in which control of epithelial cell proliferation in and around periodontal tissue is desired.

Yet another aspect of the present invention relates to the use of therapeutic preparations to control hair growth. For example, certain therapeutics (e.g., antiproliferative forms) can be employed as a way of reducing the growth of human hair as opposed to its conventional removal by cutting, shaving, or depilation. For instance, the present method can be used in the treatment of trichosis characterized by abnormally rapid or dense growth of hair, e.g. hypertrichosis. In an exemplary embodiment, the compounds of the invention can be used to manage hirsutism, a disorder marked by abnormal hairiness. The subject method can also provide a process for extending the duration of depilation.

Moreover, because at least certain compounds of the invention will often be cytostatic to epithelial cells, rather than cytotoxic, such agents can be used to protect hair follicle cells from cytotoxic agents which require progression into S-phase of the cell-cycle for efficacy, e.g. radiation-induced death. Treatment by the subject method can provide protection by causing the hair follicle cells to become quiescent, e.g., by inhibiting the cells from entering S phase, and thereby preventing the follicle cells from undergoing mitotic catastrophe or programmed cell death. For instance, compounds of the invention can be used for patients undergoing chemo- or radiation-therapies which ordinarily result in hair loss. By inhibiting cell-cycle progression during such therapies, the subject treatment can protect hair follicle cells from death which might otherwise result from activation of cell death programs.

The subject method can also be used in the treatment of folliculitis, such as folliculitis decalvans, folliculitis ulerythematosa reticulata or keloid folliculitis. For example, a cosmetic prepration of a compound of the invention can be applied topically in the treatment of pseudofolliculitis, a chronic disorder occurring most often in the submandibular region of the neck and associated with shaving, the characteristic lesions of which are erythematous papules and pustules containing buried hairs.

Other clinical uses for the compounds of the invention include their use as anti-inflammatory compounds.

The compounds of the invention can also be used to inhibit proliferation and/or kill cells having a high Ψm. Cells having an abnormally high Ψm include cancer cells, e.g., human breast adenocarcinomas (see, e.g., Davis et al. (1985) J. Biol. Chem. 260:13844; Summerhayes et al. (1982) PNAS 79:5292; Modica-Napolitano et al. (1998) Cancer Res. 58:71; and Modica-Napolitano et al. (1990) Cancer Res. 50:7876; and Nadakavukaren et al. (1985) Cancer Res. 45: 6093). Furthermore, to increase the efficacity of treatment with the compounds of the invention, the Ψm of the target cells can be increased, i.e., the mitochondrial membrane is hyperpolarized. This will result at least in increased uptake of the compounds by mitochondria. In addition, uptake of the compounds into cells can be augmented by increasing the plasma membrane potential, i.e., by hyperpolarizing the plasma membrane. For example, Davis et al., supra, have shown that hyperpolarization of the mitochondrial membrane of CV-1 cells (green monkey kidney epithelial cells) increases accumulation of rhodamine 123 (Rh 123) and prolongs its retention, and that hyperpolarization of the plasma membrane further heightens this effect, causing the uptake by CV-1 cells to resemble that in the human breast adenocarcinoma (MCF-7) cells.

The compounds of the invention can also be used in photochemotherapy, as described, e.g., in Modica-Napolitano et al. (1990) Cancer Res. 50:7876 and in Modica-Napolitano et al. (1998) Cancer Res. 58:71. These authors decribes that photoactivation enhances the mitochondrial toxicity of the cationic rhodacyanine MKT-077. Accordingly, in one embodiment, a compound of the invention is administered to a subject having cancer, e.g., in a tumor of a subject, and the tumor is subjected to high-intensity light to increase the anti-proliferative and/or cytotoxic effect of the compounds.

Also within the scope of the invention are methods for inhibiting growth of non-mammalian cells, which have similar signal transduction pathways as those in mammalian cells. Exemplary cells include yeast cells. Accordingly, the compounds of the invention can be used as anti-fungal agents to treat fungal infections on animals, e.g., humans. The compounds can also be used for stopping fungus growth on objects, e.g., mold growth on shower curtains.

Methods of Administration of the Compounds of the Invention

The therapeutic methods of the invention generally comprises administering to a subject in need thereof, a pharmaceutically effective amount of a compound. The compounds of the invention can be administered in a “growth inhibitory amount,” i.e., an amount of the compound which is pharmaceutically effective to inhibit or decrease proliferation of target cells. The compounds can also be administered in a “differentiation modulating amount”, e.g., “differentiation-inducing amount” or “differentiation-inhibiting amount,” which is an amount of the compound which is pharmaceutically effective to modulate differentiation of target cells. The compounds of the invention can also be administered in a “cell death inducing amount,” which is an amount of a compound which is pharmaceutically effective to induce cell death of target cells. The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including intravenously, intramuscularly, intraperitoneally, subcutaneously, rectally and topically. In a preferred embodiment, one or more compounds are injected directly into a tumor of the subject to be treated.

Toxicity and therapeutic efficacy of the compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Reagents which exhibit large therapeutic indices are preferred. While reagents that exhibit toxic side effects may be used, care should be taken to design a delivery system that targets such reagents to the site of affected tissue in order to minimize potential damage to uninfected cells and, thereby, reduce side effects.

The data obtained from cell culture assays and animal studies can be used in formulating a range of dosage for use in humans. The dosage of such reagents lies preferably within a range of circulating concentrations that include the ED₅₀ with little or no toxicity. The dosage may vary within this range depending upon the dosage form employed and the route of administration utilized. For any reagent used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. For example, some compounds of the invention are effective at concentrations of 10 nM, 100 nM, or 1 μM. Based on these numbers, it is possible to derive an appropriate dosage for administration to subjects.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of the invention are employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The compounds identified by the instant method may also be co-administered with other well known therapeutic agents that are selected for their particular usefulness against the condition that is being treated. For example, the instant compounds may be useful in combination with known anti-cancer and cytotoxic agents. Similarly, the instant compounds may be useful in combination with agents that are effective in the treatment and prevention of neurofibromatosis, restinosis, polycystic kidney disease, infections of hepatitis delta or HPV and related viruses and fungal infections. The instant compounds may also be useful in combination with other inhibitors of parts of the signaling pathway that links cell surface growth factor receptors to nuclear signals initiating cellular proliferation.

Drugs can be co-administered to a subject being treated with a compound of the invention include antineoplastic agents selected from vinca alkaloids, epipodophyllotoxins, anthracycline antibiotics, actinomycin D, plicamycin, puromycin, gramicidin D, taxol, colchicine, cytochalasin B, emetine, maytansine, or amsacrine.

Classes of compounds that can be used as the chemotherapeutic agent (antineoplastic agent) include: alkylating agents, antimetabolites, natural products and their derivatives, hormones and steroids (including synthetic analogs), and synthetics. Examples of compounds within these classes are given below. Alkylating agents (including nitrogen mustards, ethylenimine derivatives, alkyl sulfonates, nitrosoureas and triazenes): Uracil mustard, Chlormethine, Cyclophosphamide (Cytoxan™), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide. Antimnetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine. Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol.RTM. and is described in more detail below in the subsection entitled “Microtubule Affecting Agents”), Mithramycin, Deoxycoformycin, Mitomycin-C, L-Asparaginase, Interferons (especially IFN-a), Etoposide, and Teniposide.

Hormones and steroids (including synthetic analogs): 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex. Synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA).

If formulated as a fixed dose, such combination products employ the combinations of this invention within the dosage range described below and the other pharmaceutically active agent(s) within its approved dosage range. Combinations of the instant invention may also be used sequentially with known pharmaceutically acceptable agent(s) when a multiple combination formulation is inappropriate.

Radiation therapy, including x-rays or gamma rays which are delivered from either an externally applied beam or by implantation of tiny radioactive sources, may also be used in combination with an inhibitor of prenyl-protein transferase alone to treat cancer.

When a composition according to this invention is administered into a human subject, the daily dosage will normally be determined by the prescribing physician with the dosage generally varying according to the age, weight, and response of the individual patient, as well as the severity of the patient's symptoms.

Additional Uses for the Compounds of the Invention

As further described in the Examples, at least two compounds (F16 and QR) create a fluorescence in cells that are contacted with it and which are cells that will be killed by the compound. Thus, at least some compounds of the invention can be used as markers of cells that are susceptible of being killed or cell cycle arrested by the compounds or of cells which are being killed or cell cycle arrested. For example, it may be useful to be able to identify a cell that is being killed or cell cycle arrested during a treatment, e.g., for monitoring purposes during the treatment. In particular, because cells that have been killed may be discarded by the body, it may be difficult to assess exactly the effect of the compound. In the case of compounds which inhibit cell proliferation, these are also particularly useful, since it may not otherwise be possible to identify cells which have been cell cycle arrested.

Based on the structure of the compounds identified, in particular, the structure of E20, at least some of the compounds may be nucleic acid interchalating agents. Thus, at least certain of the compounds of the invention could be used as interchalating agents.

Also within the scope of the invention are in vitro applications for compounds which fluoresce. These compounds can be used, e.g., in cell culture.

As indicated above, the compounds are also expected to be useful in inhibiting proliferation and/or killing non-mammallian organisms, e.g., yeast. The compounds can be used, e.g., as antifungal agents.

Kits of the Invention

All the essential materials and reagents required for administering the compounds of the invention may be assembled together in a kit. When the components of the kit are provided in one or more liquid solutions, the liquid solution preferably is an aqueous solution, with a sterile aqueous solution being particularly preferred.

For in vivo use, as discussed below, the compound may be provided in combination with one or more other drugs, e.g., chemo- or radiotherapeutic agent. These normally will be a separate formulation, but may be formulated into a single pharmaceutically acceptable composition. The container means may itself be geared for administration, such as an inhalant, syringe, pipette, eye dropper, or other such like apparatus, from which the formulation may be applied to an infected area of the body, such as the lungs, or injected into an animal, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent and, e.g., explaining how the agent will decrease proliferation of cells.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with a separate instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of compound levels or reactions in vitro.

The present invention is further illustrated by the following examples which should not be construed as limiting in any way. The contents of all cited references (including literature references, issued patents, published patent applications as cited throughout this application) are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold

Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Preparation of Activated Neu-Overexpressing Cells

This example describes the preparation of cells constitutively expressing activated Neu oncogene.

EpH4 cells, which are non-transformed mouse mammary epithelial cells, which grow in a monolayer exhibiting the characteristic “cobble stone” shape of epithelial cells were chosen for stable transfection with the Neu oncogene. EpH4 cells were described by Reichmann (1992) Cell 71:1103 as a subclone of a mixed epithelial /mesenchymal polyclonal line originally reported in Reichman et al. (1989) J. Cell Biol. 108:1127. EpH4 is an acronym for epithelial (Ep) hygromycin (H) clone 4 (4). These cells are also described, e.g., in Lopez-Barahona et al. EMBO J (1995) 14(6):1145-55. EpH4 cells were grown in Dulbecco's modified Eagle's medium (DMEM), 5% FBS at 37° C./5% CO₂.

EpH4 cells stably expressing the full length rat Neu coding sequence were prepared as follows. The Neu transforming oncogene (NeU^{T}) cDNA (GenBank Accession NoX03362) was subcloned into the plasmid pRETRO-OFF (Clontech) as follows. A SalI/XhoI/HindIII/NotI adapter was introduced into pBluescript (Stratagene). The HindIII/SalI fragment containing full length Neu^{T} cDNA was excised from a vector, which itself was derived from pSV2neuNT (Bergman (1986) Nature 3190:226) and inserted into corresponding sites in p-Bluescript. Finally the chimeric vector carrying Neu^{T} cDNA was subjected to restriction digestion with NotI, and the excised 7 kb fragment including the full length Neu^{T} coding sequence and SV40 polyadenylation sequence was introduced into the NotI site in pRETRO-OFF.

EpH4 cells were stably transfected with the empty pRETRO-OFF vector or with the pRETRO-OFF vector in which the Neu^{T} cDNA was inserted, using Fugene Reagent according to manufacturer's protocol (Boehringer Mannheim). Stable clones were isolated by selection on puromycin at 1.2 μg/ml. Several stably transfected clones were obtained. One clone having stably integrated the vector, and referred to as “EpH4-EV” and two clones having stably integrated the vector with the Neu^{T} cDNA, referred to as “EpH4-A6” and “EpH4-E8,” were used to identify anti-proliferative compounds. As indicated by viewing of the cells under phase contrast microscopy, Neu^{T} expression in EpH4 cells alters cell morphology in two-dimensional cultures, relative to non transfected EpH4 cells and EpH4 cells transfected with an empty vector: cells expressing Neu^{T} lose their typical “cobble stone” shape.

To further confirm that the Neu^{T} was being expressed and thereby transforming the EpH4 cells, two of the major signaling pathways mediating the effects of activated Neu, the MAP kinase and the PI3K/PKB pathways, were examined. Accordingly, the protein level of pPKB, p44-42 MAPK, Neu, and β-actin (as a control) proteins were determined in Neu-transformed and control cells, as follows. EpH4, EpH4-A6 and EpH4-A8 were incubated in serum-free medium for 5 hrs and subsequently lysed in lysis buffer with protease inhibitors (40 mM HEPES, 150 mM NaCl, 10 mM sodium pyrophosphate, 1% NP40, 10 mM NaF, 2 mM EDTA, 10□ g/mL aprotinin, 10 □M leupeptin, 10 □ g/mL pepstatin, 10 □M EP475, 1□M PMSF, 10 □M Na₃VO₄.). The lysate was spun at 140,000×g for 30 min to separate the insoluble material. Protein concentration was determined on the supernatant using Bradford reagent (Bio-Rad). An aliquot of each lysate containing equivalent amount of protein was separated by SDS-PAGE and transferred to Immobilon-P membranes (Millipore). The transfer buffer consisted of 25 mM Tris, 190 mM glycine, 20% methanol, 0.005% SDS. Membranes were blocked with 1% BSA in 150 mM NaCl, 20 mM Tris-HCl pH 7.4, 0.3% Tween 20 (TBST), and the primary antibodies (1□ g/mL) and secondary antibodies were in 0.2% BSA in the same buffer. Anti-phospho MAP kinase and anti-phospho PKB were purchased from New England Biolabs. Anti-phospho Neu was purchased from Upstate Biotechnology Inc. Membranes were washed with TBST buffer. In all cases blots were developed with horseradish peroxidase conjugated to anti-rabbit IgG and to anti-mouse IgG (Amersham). The enhanced chemiluminescence reagent (Pierce) was used for detection.

The results indicate that an increase in the activated form of MAP kinase and PKB was observed in the Neu-overexpressing cells using antibodies that specifically recognize the phosphorylated active form of the proteins. The results also indicate that the Neu protein is expressed in EpH4-A6 and -A8 and is phosphorylated, as expected, but is not detectable in EpH4 cells or in EpH4-EV cells. It was also shown that these Neu-overexpressing cells form fast growing tumors when injected into nude mice, whereas as the non-expressing cells did not, further confirming that they are transformed cells.

Thus, Neu is capable of active signaling in the EpH4 cells, providing a good experimental system to utilize in a high-throughput screen for inhibitors of Neu-mediated growth.

### Example 2

### Identification of Compounds which Inhibit Proliferation of Activated Neu-overexpressing Cells

This example describes the identification of six compounds which inhibit proliferation of activated Neu-tranformed EpH4 cells.

In order to look for selective inhibitors of cell proliferation dependent on Neu, a chemical library was screened using a cytoblot assay. The ability of small molecules to inhibit cell proliferation at a final concentration of 10 μwas determined by the relative incorporation of BrdU into DNA in EpH4-A6 Neu-overexpressing and EpH4-EV control cells.

A modified version of a protocol previously described was used for the cytoblot assay (Stockwell et al. (1999) Chem Biol 6(2), 71-83). Briefly, for this cytoblot assay, 2,000 control (EpH4-EV) and Neu overexpressing (EpH4-A6) cells were seeded in 40 μL of growing medium in duplicate in each well of a white 352-well plate (Costar). 16,000 compounds from a chemical library (Chembridge) were transferred to each well to a final concentration of 10-15 μM. Plates were incubated at 37° C. with 5% CO₂ for 36 hours. BrdU was then added to a final concentration of 20 μM and the cells were incubated at 37° C. with 5% CO2 for an additional 3 hours. Cells were then washed with PBS and fixed with 75% ethanol/25%TBS at 4° C. for 1 hour. DNA denaturation was achieved by incubation with 2M HCl/0.5% Tween-20 for 20 min, followed by neutralization with 2M NaOH in Hank's Balanced Salt Solution (HBSS). Cells were subsequently washed with HBSS, blocked with PBS/0.3% Tween-20/1% BSA (PSTB), and incubated with 0.5 μg/mL of mouse anti-BrdU antibody (PharMingen) and 1:5,000 dilution of anti-mouse IgG coupled to HRP in PBSTB for 1 hour at room temperature. Finally the cells were washed with PBS and 20 μL of 1:1 mix of the enhanced chemiluminescence reagent (Pierce) was then added to each well of a plate. In every case addition of reagents was done using a mutli-drop 384 liquid dispenser (Labsystems) and the washes using a plate washer (Bio-Tek Instruments). Chemiluminescent signal from each plate was detected by autoradiograph and multilabel counter Victor² (Wallac) in order to quantitate results. A control cytoblot using inhibitory anti-Neu monoclonal antibodies was performed following the same procedure with the exception that anti-Neu antibody was added to the medium at a final concentration of 20 μg/mL. A small molecule inhibitor is identified by a decrease in BrdU incorporation in EpH4-A6 cells relative to control EpH4-EV cells.

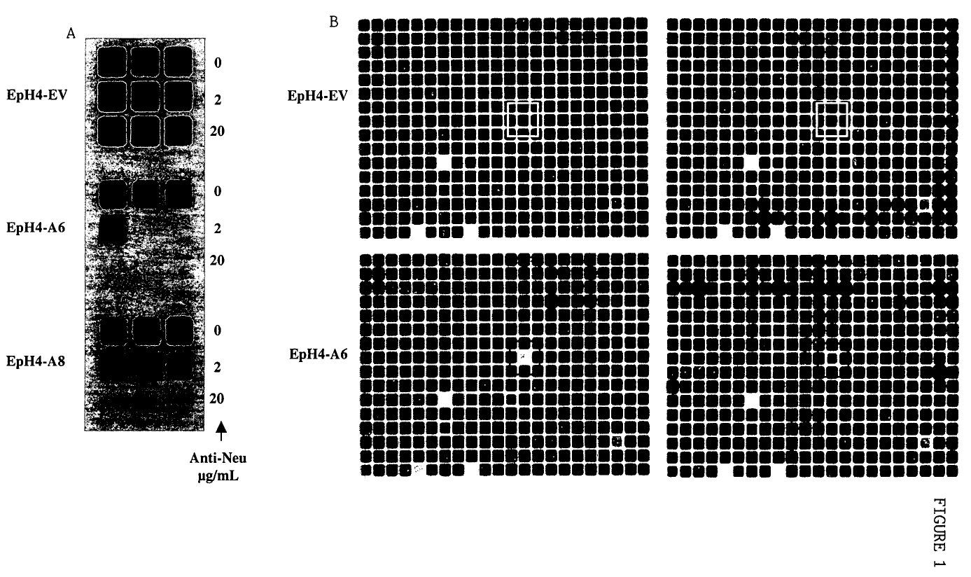

The results for one particular set of 4 plates (containing duplicates of two plates) from the 51 sets tested during the screen are shown in FIG. 1. In this particular case, several small molecules exhibited an anti-proliferative effect on Neu-expressing cells that is indistinguishable from that exhibited on control cells. However, in one instance a small molecule selectively affected proliferation of the transformed cells, i.e., did not affect proliferation of the control cells (see bracketed slot in the plates in FIG. 1B).

Out of the 16,000 compounds comprising the chemical library, 75 compounds were identified as being potentially interesting. Of these 75 compounds, 55 compounds were identified as exhibiting anti-proliferative activity for EpH4-A6 cells. The other compounds had anti-proliferative activity also against EpH4-EV.

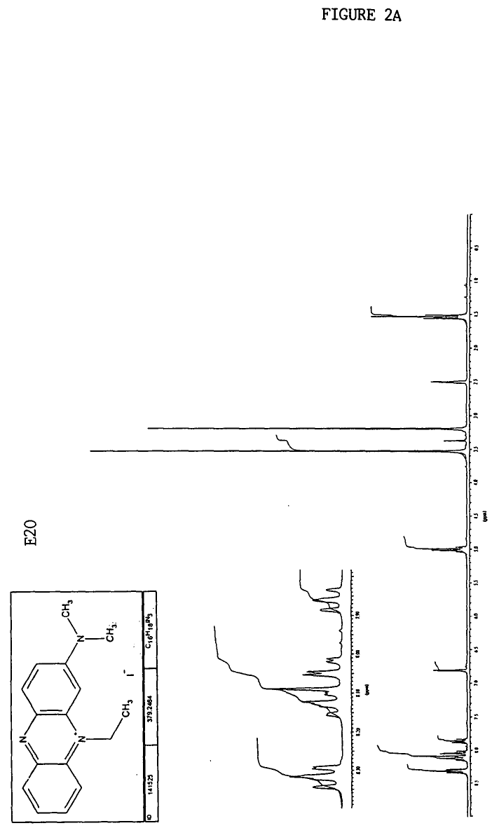

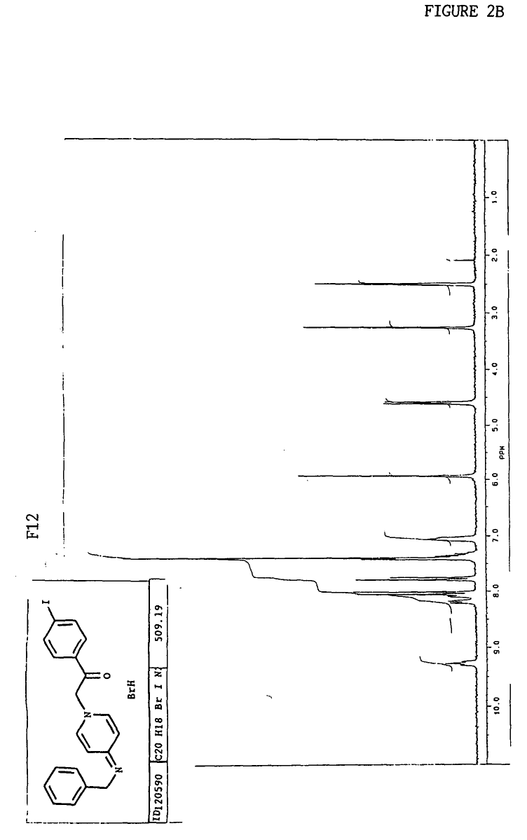

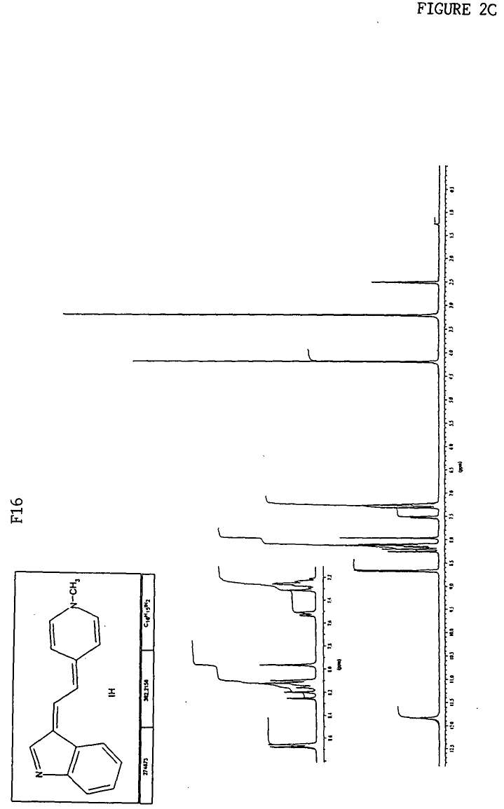

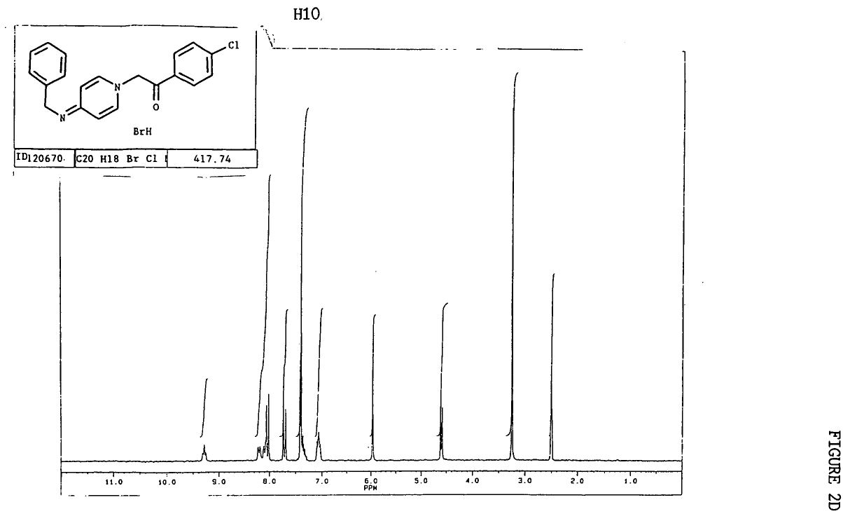

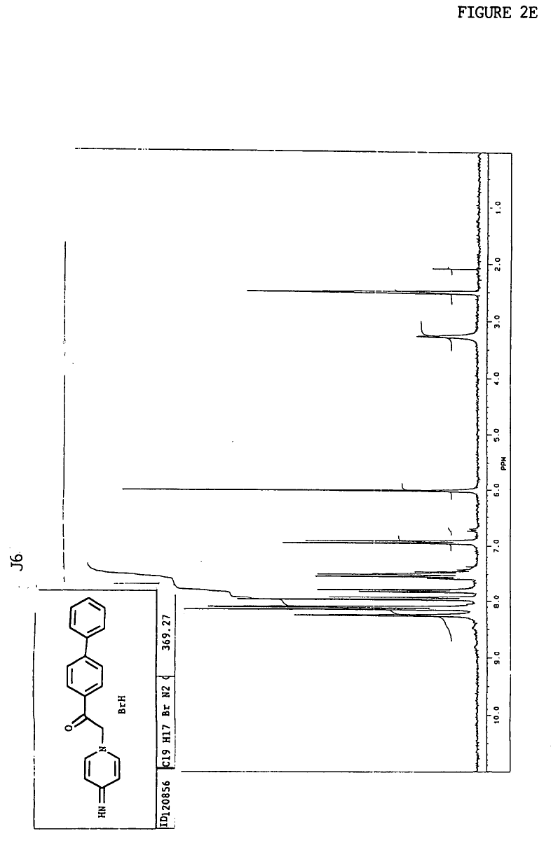

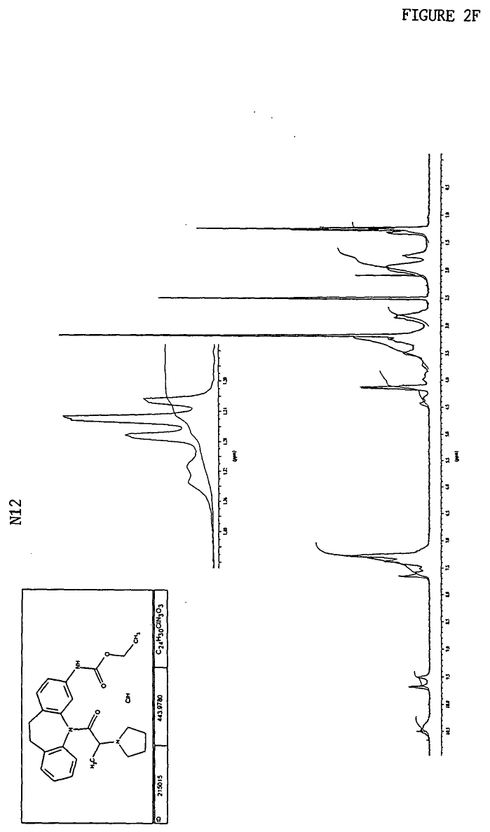

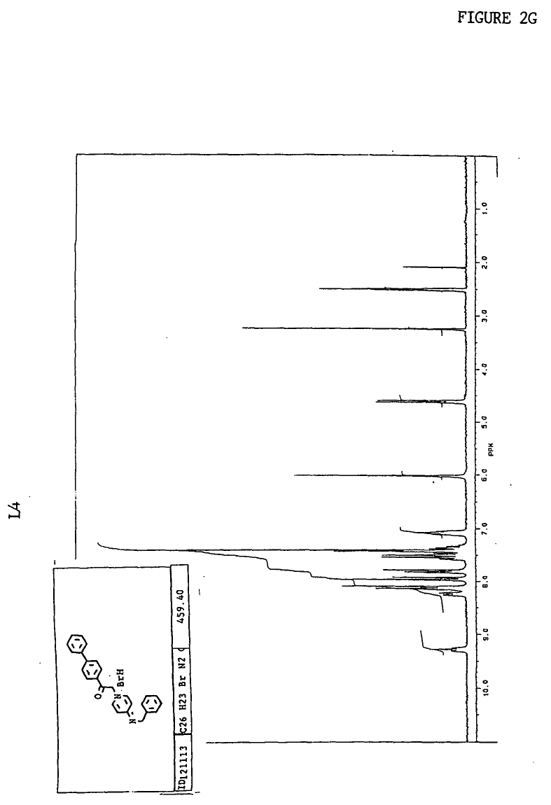

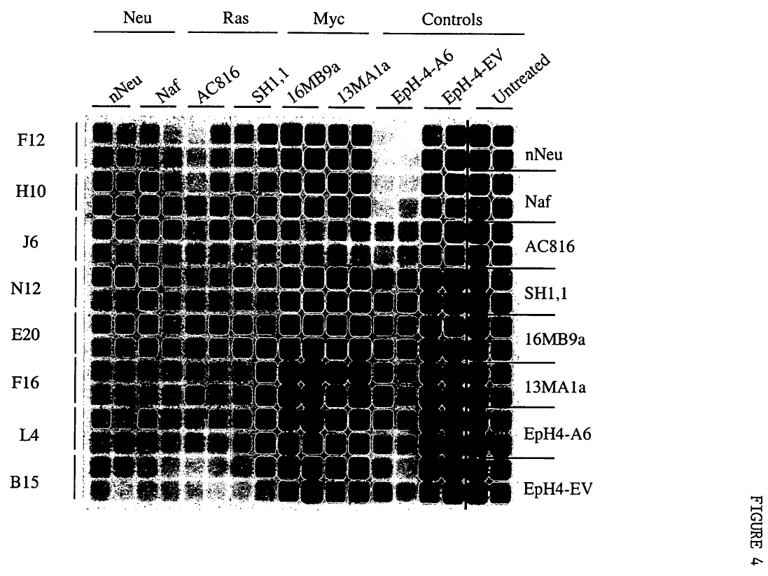

The potency of these 55 small molecules was then evaluated at lower concentrations, i.e., at 10 μM and 1 μM using the cytoblot assay described above. Among these 55 compounds, 18 showed anti-proliferative activity at concentrations as low as 10 μM and out of these, six compounds were active at concentrations as low as 1 μM. These six compounds are referred to as E20 (ID 141525); F12 (ID 120590); F16 (ID 274873); H10 (ID 120670); J6 (ID 120856); N12 (ID 215015), and their structure is shown in FIGS. 2 A-F, respectively. F16 was able to partially inhibit BrdU incorporation in EpH4-A6 cells at a concentration as low as 10 and 100 nM. Two compounds that were found to be active at about 10 μM are set forth in FIGS. 2G and H, and their antiproliferative effect is shown in FIG. 4. These two compounds are referred to as L4 (ID 121113); B15(ID217496) and QR. FIGS. 2 A-H also show proton NMR spectra of each of the molecules that confirmed their identity.

### Example 3

### Dose-Response Curve of the Antiproliferative Effect of the Compounds

In order to accurately compare the efficacy of the six small molecules identified above (E20 (ID 141525); F12 (ID 120590); F16 (ID 274873); H10 (ID 120670); J6 (ID 120856); and N12 (ID 215015)), a dose-response curve was performed using the cytoblot assay described above and different concentrations of each of the six compounds (see FIG. 3). As shown in FIG. 3, compound F16 was particularly potent in inhibiting proliferation, being effective at concentrations as low as 100 nM and even 10 n M.

Interestingly, the F16 compound creates a fluorescence in the cells that are sensitive to its anti-proliferative or cell death inducing activities at concentrations in the same range as those that induce its anti-proliferative or cell death inducing activity.

### Example 4

### Effect of Neu-Inhibiting Small Molecules on Cell Lines Transformed by other Oncogenes

The effect of these six compounds (E20 (ID 141525); F12 (ID 120590); F16 (ID 274873); H10 (ID 120670); J6 (ID 120856); and N12 (ID 215015)), as well as that of the L4 and B 15 compounds, was examined on neu, c-myc or v-Ha-ras initiated tumor cell lines.

The compounds were incubated at 10 μM concentration with tumor cell lines expressing Neu (nNeu and Naf), Ras (AC816 and SH1,1), or Myc oncogene (16MB9a and 13MA1a) or with EpH4-A6 or EpH4-EV cells as controls. These tumor cell lines are described in Guy et al. (1996) J. Biol. Chem. 271:7673; Muller et al. (1988) Cell 54: 105; Sinn et al. (1987) Cell 49:465 (SH1.1 and AC816); Stewart et al. (1984) Cell 38:627; and Leder et al. (1986) Cell 45:485, respectively. The results, which are shown in FIG. 4, show that the 6 compounds were also active on Neu and Ras tumor cell lines at 10 μM. In the case of myc tumor cell lines, only compound J6 caused a decrease in BrdU incorporation.

Thus, several selective inhibitors of proliferation of Neu-transformed cells that are effective in the nanomolar and micromolar range have been identified. These same compounds showed anti-proliferative activity on a panel of tumor cell lines at 10 μM. The fact that cell lines originated from ras- and myc-initiated tumors are also sensitive to at least some of the compounds identified in the screen suggests that the targets for these molecules could participate in essential signaling pathways utilized by a number of tumor cells. Accordingly, these inhibitors have a broad spectrum of applications.

### Example 5

### Effect of F16 on Mouse Tumor and Human Cancer Cell Lines

This Example shows the effect of F 16 on the proliferation of several human and mouse cell lines, and in particular, that the strongest anti-proliferative effect of F 16 is on breast tumor lines.

The following panel of mouse tumor cell lines derived from Neu, Ras and Myc transgenic mice and panel of human cancer cell lines were used: a) Neu-initiated mouse tumor cell lines NF980, NF324-2A, NF324-1B, SMF, NAF, Neu4145, n-Neu, Ras-initiated mouse tumor cell lines SH1,1, AC236, AC711, AC816, ACp53⁻#1, ACp53⁻4782, AC222, AC99, AC260, ACp53⁻#19, ACp53⁻#16, AC/Balb6.6, AC/Balb14, AC/Balb12 and Myc-initiated mouse tumor cell lines 16MB9a, 13MA1a; and b) human breast tumor cell lines MDA-MB231, MDA-MB435, MDA-MB436, MDA-MB453, MDA-MB-468, SKBR-3, MCF-7, T47D, ZR75; human prostate cancer cell lines LNCaP, PC-3, DU145 and the human ovarian cancer cell line SKOV-3.

For each cell line, 10⁵ cells were seeded in 4×6 well plates. Next day, F16 was added at 3 μM to the medium of 2 of the plates, while the other two were left untreated (control). Cells were counted 3 days and 7 days after initiation of treatment.

The results are shown in Tables 1 and 2. A positive scoring in these Tables represent cells in which a 2-5 and a 10-40 fold reduction in the total number of cells relative to the control untreated cells was observed after 3 and 7 days of culture with F16, respectively.

TABLE 1
Anti-proliferative effect of F16 on mouse Neu-, Ras-,
or Myc-initiated tumor cell lines

| Oncogene | Cell Line | Tumor of origin | F16 response |
|---|---|---|---|
| Neu | NF980 | Breast | + |
| | SMF | Breast | + |
| | NAF | Breast | + |
| | n-Neu | Breast | + |
| | Neu4145 | Breast | + |
| | NF324-2A | Breast | + |
| | NF324-1B | Breast | + |
| Ras | AC/Balb12 | Fibrosarcoma | − |
| | AC/Balb14 | Fibrosarcoma | − |
| | AC/Balb6.6 | Fibrosarcoma | − |
| | AC/p53⁻#16 | Fibrosarcoma | − |
| | AC/p53⁻#19 | Fibrosarcoma | − |
| | AC260 | Jaw | − |
| | AC99 | Neck | − |
| | AC222 | Int. | − |
| | AC/p53⁻4782 | Int. | − |
| | AC/p53⁻#1 | Salivary | − |
| | AC816 | Breast | + |
| | AC711 | Breast | + |
| | AC236 | Breast | + |
| | SH1.1 | Breast | + |
| Myc | 16MB9a | Breast | − |
| | 13MA1a | Breast | − |

TABLE 2
Anti-proliferative effect of F16 on human cancer cell lines

| Cell Line | Tumor of origin | F16 response |
|---|---|---|
| SKBR-3 | Breast | + |
| T47D | Breast | + |
| ZR75 | Breast | + |
| BT474 | Breast | + |
| MCF-7 | Breast | + |
| MDA-MB-231 | Breast | − |
| MDA-MB-435 | Breast | − |
| MDA-MB-436 | Breast | + |
| MDA-MB-453 | Breast | + |
| MDA-MB-468 | Breast | + |
| DU145 | Prostate | − |
| LNCaP | Prostate | − |
| PC3 | Prostate | − |
| SKOV-3 | Ovary | − |

These results showthat, among the mouse cell lines tested, those derived from breast tumors from Neu and Ras transgenic mice exhibited a marked sensitivity for F16. Eight of the ten cell lines of the human breast cancer panel were also affected by the small molecule. To the contrary, the growth of the mouse fibrosarcoma cell lines derived from ras-initiated mice was not affected. It has also been shown that F16 has to some extent selectivity for β-catenin-initiated mouse breast tumor cell lines. Thus, the results show the selectivity of F16 towards breast tumor cell lines, and demonstrate that the anti-proliferative activity of F16 is not limited to cells of mouse origin, but also affects human cells.

### Example 6

### Cell Cycle Arrest and Increased Apoptosis in F16-Treated Cells

This Example demonstrates that F16 causes a decrease in the number of cells in the S phase of the cell cycle and an increase in the number of cells in the G1 and sometimes G2 phases of the cell cycle.

The percentage of cells in the different phases of the cell cycle was assessed by measuring BrdU incorporation into the DNA of the cells. BrdU incorporation into DNA of untreated or F16 treated (3 μM/30-36 hr) EpH-EV and EpH-A6 cells was assessed by FACS as follows. After F16 or mock treatment, the cells were pulsed with BrdU (20 μM) for 1 hour and then fixed in 70% ethanol at −20° C. overnight. Next morning cells were washed twice with 1×PBS and the DNA was denatured by incubation of the cells in 2N HCl/0.5% Triton X-100. The cells were then washed twice in 0.1M Na₂B₂O₇, and once in PBS. The cells were resuspended in PBS, incubated with 20 μL of anti-BrdU/FITC(Becton Dickinson) for 1 hour and subsequently washed with 1×PBS and resuspended in PBS. RNA was digested with RNaseA (1 μL of 10 mg/mL stock) at 37° C. for 30 minutes. Finally, the cells were diluted with 1 mL of 5 μg/mL propidium iodide/PBS and incubated on ice for 15 minutes. The labeled cells were analyzed using the FACSCalibur (Becton Dickinson).

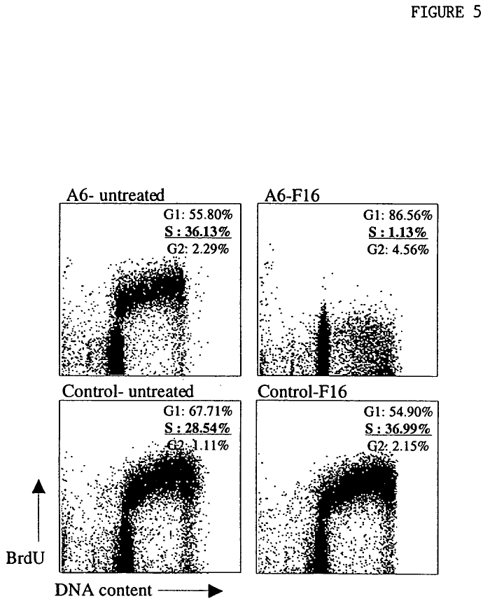

FIG. 5 shows a representative result of these studies. These studies showed that F16 causes a dramatic decrease in the percentage of cells in the S phase of the cell cycle, and an increase in the percentage of cells in G1 phase. These results are in agreement with those obtained in the cytoblot screen assay (based on BrdU incorporation).

The effect of F16 (3 μM) on BrdU incorporation into DNA of various Neu-induced, Ras-induced and Myc-induced tumor cells lines was examined using the same technique. The results, which are shown in Table 3, indicate that, except for 16MB9a, a myc tumor cell line that is not affected by F16, the other cell lines, all of which are sensitive to F16 show, as expected, a decrease in the percentage of cells in the S phase and an increase in the percentage of cells in G1 after 30 hr of incubation in medium containing F16 (3 μM). In some cases, an increase in the amount of cells in G2 phase was observed. This may reflect the heterogeneity of the tumor cells, some of which may have acquired defects in various cell cycle regulators as secondary events.

TABLE 3
Percentage of cells in G1, S, or G2 phase of
the cell cycle after treatment with F16

| Tumor cell | Without F16 | | | With F16 | | |
|---|---|---|---|---|---|---|
| line | % G1 | % S | % G2 | % G1 | % S | % G2 |
| n-Neu | 54.9 | 30.0 | 12.1 | 64.5 | 18.2 | 15.4 |
| | 55.1 | 30.7 | 11.1 | 61.5 | 19.6 | 17.8 |
| NF980 | 59.3 | 29.4 | 9.0 | 69.6 | 17.0 | 8.9 |
| | 57.0 | 35.5 | 5.5 | 68.6 | 17.3 | 12.4 |
| NAF | 80.5 | 13.9 | 1.9 | 86.8 | 4.7 | 3.6 |
| | 81.6 | 11.9 | 2.9 | | | |
| AC816 | 89.3 | 9.6 | 0.6 | 96.8 | 0.1 | 2.7 |
| | | | | 95.1 | 0.2 | 2.8 |
| SH1.1 | 75.2 | 19.9 | 3.7 | 81.3 | 7.1 | 10.4 |
| | 76.4 | 18.4 | 3.6 | 79.7 | 7.1 | 13.2 |
| Myc | 83.8 | 12.3 | 1.1 | 82.7 | 12.8 | 1.5 |
| | 82.5 | 12.6 | 1.5 | 83.8 | 12.7 | 1.5 |

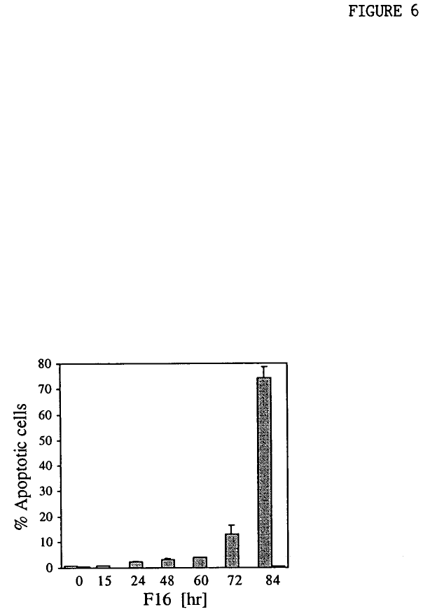

Prolonged incubation of sensitive cells with 3 μM F16 resulted in increased cell death. A time course incubation of A6 and control EV cells in F16-containing medium resulted in increasing number of apoptotic cells as evidenced by DNA fragmentation, propidium iodided staining and cytochrome c release from mitochondria to the cytosol (see FIG. 6). The DNA fragmentation assay was conducted by isolating DNA from 1×10⁶ of untreated or F16 treated for 48, 60 and 72 hr using Suicide-Track TM (Oncogene) according to manufacturer's procedure. Samples were resolved by agarose gel electrophoresis and ethidium-bromide stained. Cytochrome c release was measured by immunofluorescence as follows. Untreated or F16-treated A6 cells were washed and fixed with ice-cold 3.7% paraformaldehyde at RT, permeabilized for with ice-cold 0.01% (v/v) Nonidet P-40 for 20 min and incubated with 0.5% BSA/PB. Immunodetection was performed with monoclonal anti-cytochrome c antibody (PharMingen) and secondary anti-mouse-TRITC conjugated antibody. Cells were photographed under Axioskop using a Spot Camera (Diagnostic Instruments).

### Example 7

### Effect of F16 in Vivo

As a first step towards evaluating the potential effect of F16 in vivo, pharmacokinetics studies with F16 were performed in nude mice.

Based on the literature, molecules of similar size to that of F16 have been injected intraperitoneally at a dose of 5 to 50 mg/kg of body. Based on results with F16, 50 mg/kg might be close to the maximum tolerable dose (MTD), since one mouse out of 5 mice injected with 50 mg/kg of F16 died a few hours after the injection. Two other mice looked letargic after the injection, but then recovered. Thus, a lower amount of F16 was used in this Example.

To determine the levels of F16 present in the blood of mice after injection of F16, two mice were injected intraperitoneally with 25 mg/kg F16 and two mice were injected intraperitoneally with 12.5 mg/kg of F16. The level of F16 in the serum of these mice was determined at 15 minutes, 30 minutes, 1 hour, 3½ hours and 7 hours after the injection of F16, as follows. 40-50 μL of blood was obtained from the tail vein of the mice and placed into tubes incubated in ice and containing Heparin to a final concentration of 20U/mL of blood. Samples were centrifuged at 6,000 rpm for 3 minutes in the cold room. Serum was transfered to a clean tube and stored at −70° C. until further processing. 2 μL of serum samples were spotted onto Whatman paper, alongside with dilutions of F16 as a standard. Since F16 is a fluorescent compound (see below), the presence of F16 was deteremined by measuring its fluorescence emission using the Storm 860 (Molecular Dynamics). The concentration of F16 in serum samples was estimated by visual comparison to the intensity of the standard.

Doses of 12.5 and 25 mg/kg F16 are well tolerated by the mice. As indicated in FIG. 7, the maximum level of F16 in the blood of the mice occurred between 1-3 hours (micromolar range, compared to standard). The level of F16 decreased considerably (nanomolar range, compared to standard) at 7 hours after the injection. F16 was not detected in blood 20 hours after the injection.

Based on these results, a dose of 20 mg/kg twice a day was used to assess the effect of F16 on tumor growth in nude mice. Twenty nude mice of 8-10 weeks of age were injected subcutaneously with 10⁶ EpH-A6 cells in the left and right flanks. Three days after the cell injection, a 20 mg/kg dose of F16 (40% DMSO/60% PBS) was injected intraperitoneally twice a day to a group of 10 mice, and an equivalent dose of vehicle to the remaining 10 mice (placebo mice). After 10 days of treatment, tumors were removed from all mice and weighted. Tumor tissue was used for FACS analysis, pathology, and some were stored at −70° C. until further processing.

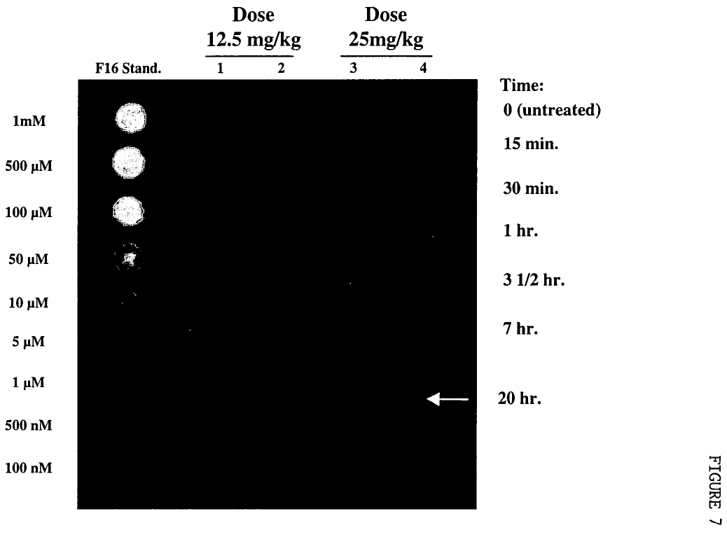

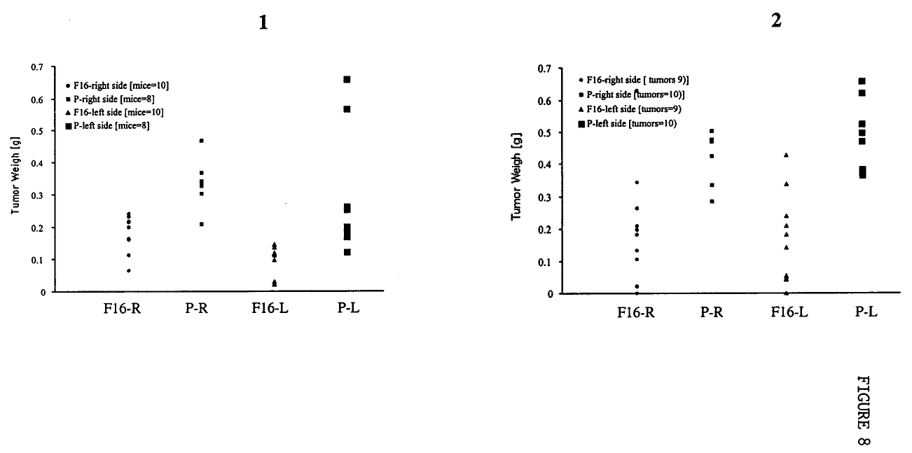

FIG. 8 and Table 4 shows the weight of the tumors of the control and F16 treated mice. These results indicate that a 2-3 fold difference in tumor size was observed in the F16 treated group relative to the control mice. The experiment was repeated with a second group of 20 mice, and similar results were obtained.

TABLE 4
Tumor weights in mice treated with F16 and in control mice

| Right flank | Right flank | Left flank | Left flank |
|---|---|---|---|
| F16 | Placebo | F16 | Placebo |
| Experiment #1¹ | | | |
| 0.2412 | 0.836 | 0.097 | 0.2518 |
| 0.2408 | 0.336 | 0.1163 | 0.1686 |
| 0.2174 | 0.4698 | 0.1174 | 0.1905 |
| 0.214 | 0.3261 | 0.1131 | 0.1217 |
| 0.0666 | 0.3669 | 0.0293 | 0.5667 |
| 0.1984 | 0.341 | 0.1365 | 0.2621 |
| 0.1597 | 0.2079 | 0.021 | 0.6589 |
| 0.1645 | 0.3012 | 0.1463 | 0.1982 |
| 0.1127 | | 0.1127 | |
| 0.2314 | | 0.1186 | |
| Experiment #2² | | | |
| 0.022 | 0.4264 | 0.054 | 0.4965 |
| 0.632 | 0.4716 | 0.4294 | 0.7122 |
| 0.2078 | 0.5028 | 0.3368 | 0.3841 |
| 0.1798 | 0.7983 | 0.041 | 0.6206 |
| 0.1063 | 0.3336 | 0.2375 | 0.5253 |
| 0.2621 | 0.2823 | 0.2087 | 0.3639 |
| 0.1315 | 0.4757 | 0.1809 | 0.6565 |
| 0.1971 | 0.7691 | 0.0454 | 0.4692 |
| 0.3451 | 0.2574 | 0.1417 | 0.6236 |
| 0 | 0.2598 | 0 | 0.693 |

¹Two placebo mice died for unknown reasons during experiment.
²10 mice were included in each group, but only nine F16 treated mice showed tumors.

The total weight of the mice was also monitored every other day during the course of the treatment with F16. Placebo mice had an average total body weight gain of about 3.2%, whereas F16 treated mice had an average total body weight loss of 3.3%, relative to their weight at the beginning of the experiment.

All the tumors from the twenty mice as well as kidney, liver and heart samples from four placebo and four F16 treated mice were sent for pathology examination. The results show no major differences in the kidney, heart and liver samples between F16 and placebo treated mice. Examination of the tumors indicated that the degree of necrosis in the placebo mice was higher than that in the tumors from the F16 treated mice, which correlate with the bigger tumor mass in the placebo mice. Necrosis probably results from the lack of sufficient blood supply to these fast growing tumors. A decrease in number of cells undergoing mitosis was observed in at least some of the F16 treated tumors when compared to untreated tumors, indicating inhibition of cell proliferation by F16.

Several tumor cell populations from F16 treated and untreated mice were analyzed by FACS, in order to determine the presence of F16 in the cells. The results, which are shown in FIG. 8, and which are based on the fluorescent signal detected in tumors of F16-treated animals, indicate that F16 is delivered to the tumor cell mass.

Thus, the results indicate that F16 is effectively delivered to the tumor site, and that it reduces tumor growth, probably due at least to inhibition of cell proliferation.

### Example 8

### Intracellular Location of F16

This Example shows that living cells that are not affected by F16 do not take up F16. This Example further shows that, when taken up by living cells, F16 localizes to the mitochondria and to nucleolar particles of the cell.

F16 is a fluorescent compound with a maximum absorption of approximately 420 nm, and a maximum emission of approximately 520 nm, which allows for visualization of its fluorescence signal. This property of F16 was used to determine the fate of the compound once inside the cell. F16 used in this Example and in Examples 5-7 was obtained from Interbioscreen Ltd., Moscow, Russia (catalogue number 2S-13965) and from Asinex Ltd., Moscow, Russia (catalogue number BAS 1027104).

The following cell lines were used in this Example: EpH4-A6 (Neu^{T}); EpH4-EV; Neu-initiated mouse tumor cell lines NF980, NAF and nNeu; Ras-initated tumor cell lines SH1.1 and AC816; a Myc-initiated tumor cell line 16MB9a; and normal mammary epethileal cells HC11, NuMg and EpH4. The cells were grown on 60 mm plates and incubated with medium alone or medium containing 3 μM F16 for 24 hours at 37° C. The fluorescence signal of F16 was visualized under inverted microscope Axiovert (Zeiss) using a standard fluorescein filter set. Cells were photographed using a digital camera D1 (Nikon).

The results, which are shown in FIG. 8 indicate that EpH4-EV cells that are unaffected by F16 do not show any uptake/retention of the compound, whereas EpH4-A6 cells, ras- and neu-initiated tumor cell lines, which are sensitive to F16, show very intense fluorescent green punctuated staining. Similarly, 16MB9a, which expresses c-Myc, and which is essentially insensitive to F16, does not show fluoresence. Thus, in general, cells that are unaffected by F16 do not take up/retain F16.

In order to further refine the localization of the signal, dyes that stain specific cell compartments were used. Since the staining pattern of F16 suggested that F16 might be targeted to the mitochondria and the lysosomes, stains specific for these cell compartments were used.

2×10⁴ EpH4-EV and EpH4-A6 cells were seeded in 6 well chamber slides. Next morning, cells were left untreated or treated with F16 at 3 μM for 2 hours. Cells were incubated with Hoescht stain for 30 minutes and then visualized under the microscope. Hoescht stain specifically stains the nuclei of cells. A mitochondrial specific stain, Mitotracker Red (Molecular Probes), was added to the medium to stain mitochondria, and Lysotracker Red to stain lysosomes and acidic compartments, according to manufacturer procedure. Hoescht stain was also included in the medium (together with the other stains). Cells were photographed under Axioskop (Zeiss) using a Spot Camera (Diagnostic Instruments).

The results clearly demonstrate that F16 staining overlapped with the mitochondrial staining, but not with the lysosomal staining. In addition, some dense nuclear particles, potentially the nucleoli, were stained by F16.

These results indicate that the mitochondria were the major F16-concentrating organelle in the cell and tht some F16 is likely to be taken up by the nuclei of cells.

### Example 9

### Selective Accumulation of F16 in the Mitochondria is Driven by their Negative Transmembrane Potential

A group of molecules collectively known as delocalized lipophilic cations or DLCs that, like F16, selectively accumulate in the mitochondria of tumor cells had been previously described (Modica-Napolitano and Aprille JR. (2001) Adv Drug Deliv Rev (49) 63-70). Rhodamine 123, the thiopyrylium AA-1 and the rhodacyanine MKT-077 all share a hydrophobic structure and a delocalized positive charge that allows for their diffusion through lipid membranes and their accumulation. In that regard, F16 chemically resembles these DLCs.

In order to test the contribution of the mitochondrial membrane potential (ΔΨm) to the accumulation of F16 in mitochondria, the effect of FCCP, an uncoupler of mitochondrial respiration, and the effect of valinomycin, a potassium ionosphore, were tested.

The effect of the protonophore FCCP on F16 accumulation was performed as previously done for the colocalization experiments described above. EpH4-A6 cells preincubated with F16 (3 μM) were subsequently treated with 5 μM FCCP. Images were acquired immediately before, 1 min and 5 min after FCCP addition and visulized under 40× magnification.

The results indicate that preincubation of A6 cells with FCCP or valinomycin-containing medium retarded the entry and greatly reduced the overall accumulation of F16 inside mitochondria, indicating that indeed the mitochondrial potential played a major role in the take-up of F16. Furthermore, if FCCP was added to cells preloaded with F16, F16 mitochondrial staining was immediately lost and a diffuse signal throughout the cytoplasm emerged instead. This suggests that the ΔΨm is not only promoting F16 entrance but it also is acting as a retention force to drive the accumulation. In addition, preincubation of cells in the presence of high K⁺ medium (150 mM) to depolarize the plasma membrane, resulted in a delayed F16 appearance inside mitochondria. These results further suggests that the plasma membrane, as previously observed with other DLCs, might help to preconcentrate F16 in the cytoplasm of sensitive cells, which in turn moves into the mitochondrial matrix which is at a more negative potential.

To evaluate the effect of multi-drug resistance pumps on the F16-insensitive cells, inhibitors of MDR-1 and MRP1 were utilized (Lizardet al. (1994) Cell Biol Toxicol (10) 399-406). Co-incubation of F16-resistant normal mammary epithelial and human cancer cell lines with 3 μM F16 in the presence of 50 μM verapamil/1 mM probenecid did not result in F16 mitochondrial accumulation, indicating that at least in the cell lines under examination, F16 exclusion is not mediated by MDR-1 or MRP-1.

Based on these results, it was decided to compare in a semi-quantitative way the mitochondrial potential among cell lines that are sensitive or resistant to F16. For this purpose, A6 cells, control cells and various mouse tumor cell lines were subjected to flow cytometric analysis using tetramethylrhodamin methyl ester (TMRM), a probe whose accumulation in the mitochondrial matrix is proportional to the mitochondrial membrane potential. Nonyl-acridine orange, a probe that stains the mitochondrial lipid cardiolipin, was used to normalize according to mitochondria mass.

The mitochondrial membrane potential of various mouse cell lines was compared by flow cytometry, using TMRM, Molecular Probes) as the probe (REF). In all cases, cells were harvested and resuspended at 1×10⁶ cells/mL in cell culture medium containing 10 μM verapamil and 50 μM probenecid (SIGMA), MDR-1 and MRP-1 inbibitors respectively. TMRM was added to each tube to a final 50 nM. Cells were incubated at 37° C. for 20 min. and immediately analyzed by flow cytometry. In addition, cells were stained with nonyl-acridine orange (NAO) in order to estimate the mitochondrial mass in the cell lines probed with TMRM (Hiura et al. (2000) J Immunol (165) 2703-11). 1×10⁶ cells/mL cells were incubated with 2 μM NAO for 20 min. at 37° C. The fluorescent emission of NAO at 530 nm and 630 nm was collected. The ratio of green to red fluorescence best reflects the cardiolipin content of mitochondria. The results are set forth in Table 5.

TABLE 5
Comparison of Ψm in F16-sensitive and resistant cells by flow
cytometry analysis of TMRM staining

| | NAO ratio | TMRM emission | TMRM/NAO |
|---|---|---|---|
| Cell line | 530 nm/630 nm | (a.u.) | ratio |
| EV | 1/67 | 54 | 31/9 |
| 13MA1a | 1.17 | 80 | 68.4 |
| 16MB9a | 1.21 | 38 | 31.4 |
| A6 | 1.17 | 181 | 157.7 |
| NAF | 1.80 | 331 | 183.9 |
| AC 816 | 2.36 | 307 | 130.1 |

The results from these experiments showed a correlation between intensity of TMRM staining and the sensitivity towards F16. Cell lines that responded to F16 exhibited 2-6 fold higher TMRM staining than insensitive ones. Mitochondrial membrane potential was then imaged in situ using TMRM once again (Scaduto and Grotyohann (1999) Biophys J (76) 469-77).

For imaging of mitochondrial membrane potential in situ, A6 and control cells were seeded onto 25 mm-round glass coverslips at a density of 10⁴ cells/coverslip and grown for 2 days prior to measurement. Cells were then stained with 20 nM TMRM dissolved in Hank's balanced salt solution (HBSS) with 10% FCS, 10 mM Hepes and 5 μM verapamil (Sigma) for 20 min at 37° C. Coverslips were placed on the stage of a Nikon Eclipse TE300 inverted microscope equipped with a Xenon-lamp illumination system and with a Hamamatsu Orca ER 12-bit digital cooled CCD camera. Cells were excited at 530±2.5 using a Polychrome IV monochromator (Till Photonics), and emitted light was collected using a 560 longpass filter. Digital images were acquired every 30-60 sec with exposure times of 30 msec using a 60X, 1.4 NA Plan Apo oil immersion objective (Nikon). Images were acquired, stored and analyzed using Metamorph software (Universal Imaging). Specificity of the mitochondrial staining was confirmed by treating cells with the protonophore CCCP (2 μM), which caused the complete disappearance of the mitochondrial TMRM fluorescence.

The results indicate that mitochondrial TMRM fluorescence in Neu overexpressing cells is clearly higher than in cells transfected with the empty vector, further confrirming the flow cytometry analysis. The protonophore FCCP completely abolished the punctuated mitochondrial pattern of TMRM to a comparable level in A6 to that in EV cells. Thus, mitochondrial hyperpolarization of A6 cells and tumor cell lines was the most likely factor determining the differential accumulation of F16.

### Example 10

### Mitochondrial Structure and Function are Compromised in Cells Affected by F16

Electron-microscopy analysis was performed on untreated or F16-treated A6 cells, control cells and tumor cell lines SH1.1 and SMF to search for potential mitochondria lesions due to F16 accumulation. Cells grown in 35 mm dishes were left untreated or treated with 3 μM F16 for the indicated times, and fixed for 1 hr at room temperature in 0.1 M Cacodylate buffer containing 1.25% formaldehyde, 2.5% glutaraldehyde and 0.3% picric acid. Samples were then treated with 1% osmiuntetroxide/1.5% potassiumferrocyanide, followed by 1% uranyl acetate, dehydrated and epon-araldite embedded. Ultrathin sections were examined using a JEOL 1200EX microscope.

The results indicate that mitochondria were the only organelles in which ultrastructural abnormalities were evident as early as 15 hr of F16 treatment. A6 cells showed progressive swelling and disruption of mitochondrial integrity with concomitant mitochondrial membrane disruption, characteristic of apoptotic death. In the case of control cells, no abnormalities in mitochondria morphology were observed.

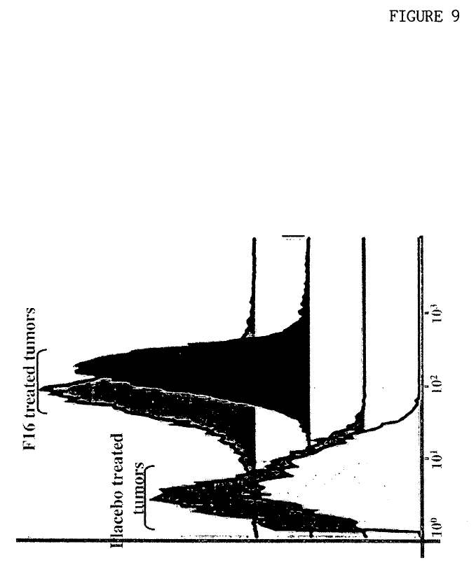

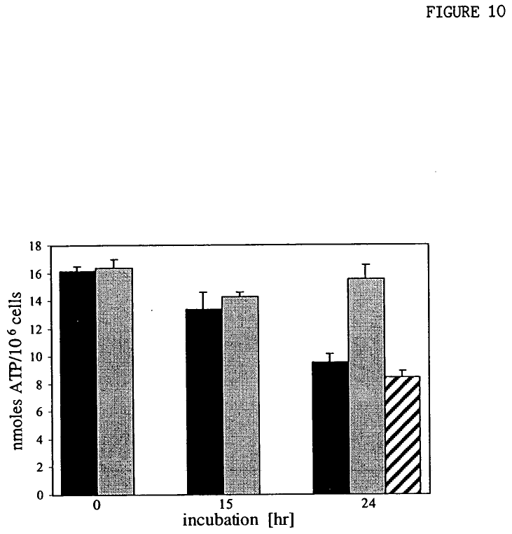

One of the major roles of mitochondria is to supply energy to the cell. To assess alterations in mitochondrial function, ATP levels were determined in F16-treated cells by the luciferase-catalyzed ATP-dependent bioluminescent oxidation of luciferin using the ATP Bioluminescence Assay Kit (Roche). EpH4-EV and EpH4-A6 cells were grown in the absence or in the presence of 3 μM F16 for 15 hr and 24 hr. As a control A6 were treated with 5 μM FCCP (Sigma). Cells were then harvested, counted and equivalent numbers used for each determination, according to manufacturer's procedure. Each determination was done in triplicate, and the entire experiment was performed twice. The results, which are shown in FIG. 10, indicate that a significant depletion in the ATP pool was observed in cells after 24 hr of F16 incubation.

Besides ATP synthesis, the mitochondria plays different roles in cell physiology, among them control of the redox state of cells. Changes in the level of superoxide anion as an indicator of alterations in reactive oxygen intermediates generation by mitochondria of cells treated with F16 was examined by flow cytometry. Changes in the levels of intracellular oxidants were investigated using the fluorescent probe dihydroethidium (DHE) (Molecular Probes). Superoxide anion can oxidate DHE to ethidium bromide (EtBr) which binds to DNA. An increase in the fluorescence of EtBr/DNA is suggestive of superoxide generation (Vanden Hoek et al. (1998) J Biol Chem (273) 18092-8). EpH4-EV and EpH4-A6 cells were grown in the absence or in the presence of 3 μM F16 for 15, 24, 48 and 72 hr. cells were resuspended and diluted to 10⁶ cells/mL. DHE was added to the cell suspension to a final 2 μM in each case. Cells were incubated at 37° C. for 15 min and immediately analysed using flow cytometry.

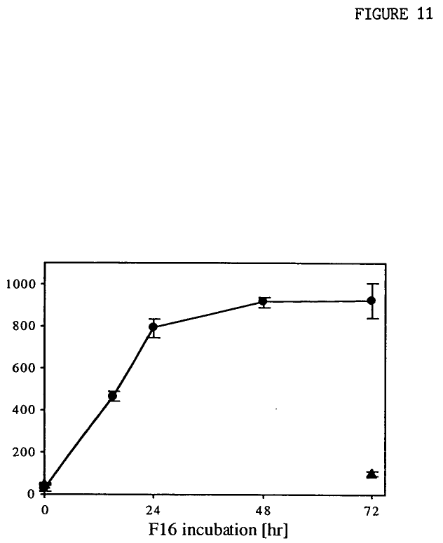

As indicated in FIG. 11, an increase in the ethidium/DNA fluorescence as a consequence of the oxidation of dihydroethidium catalyzed by superoxide was observed. This result could explain the increase in protein carbonyl content that were observed upon treatment of cells with F16. Thus, the data suggests that F16 accumulation in mitochondria correlates with an initial mitochondrial damage, which in turn compromised cellular physiology.

Thus, the data indicate that the energetic and redox balance in the F16-treated cells was drastically disrupted.

### Example 11

### Mitochondria is a Direct Target for F16-Mediated Toxicity

To understand the mode of action of F16 at the molecular level, the effect of F16 was examined on isolated mouse liver mitochondria artificially set at higher ΔΨm. Since electron micorscopy images showed that F16 induced mitochondrial swelling in the Neu-overexpressing cells, the mechanism of F16-induced swelling was investigated. It was investigated whether F16 caused opening of the permeability transition pore (PTP), a non-selective, highly controlled inner mitochondrial membrane channel that in its open conformation is permeable to solutes up to 1500 Da, ultimately leading to swelling of mitochondria (Bernardi et al. (1999) Eur J Biochem (264) 687-701).

Mitochondria were isolated from liver of Balb/c7 mice by standard differential centrifugation, and resuspended in isolation buffer (0.2 M sucrose, 10 mM Tris-MOPS pH 7.4, 0.1 mM EGTA-Tris, 0.1% delipidated BSA) (Costantini et al. (1995) Toxicology (99) 77-88). Protein concentration was determined by Bradford assay. For swelling determination, changes in side scatter at 545±2.5 nm of a 0.5 mg/ml suspension of mitochondria in experimental buffer (125 mM KCl, 10 mM Tris-MOPS pH 7.4, 1 mM Pi, 5 mM glutamate, 2.5 mM malate, 10 μM EGTA-Tris, pH 7.4) were monitored by using LS-50B spectrofluorimeter. To monitor changes in mitochondrial membrane potential, the intrinsic potentiometric properties of F16 were used. Mitochondria (0.5 mg/ml) were incubated in experimental buffer with the indicated concentrations of F16 and changes in F16 fluorescence intensity were measured, using a LS-50B spectrofluorimeter at 25° C., with excitation and emission wavelengths set at 415±2.5 nm and 520±5 nm, respectively. All reactions were performed at 25° C. and the instrument was equipped with magnetic stirring.

The results indicate that F16 caused swelling of purified mitochondria in a dose-dependent fashion, and that cyclosporin A, a specific PTP inhibitor, completely abolished this effect. The mechanism of PTP induction by F16 was further investigated as follows. PTP is a voltage dependent channel, with its open conformation being favored at lower membrane potential (Scorrano et al. (1997) Mol Cell Biochem (174) 181-4). It has previously been shown that DLCs such as chloromethyl tetramethyl rhodamine can cause mitochondrial depolarization (Scorrano et al. (1999) J Biol Chem (274) 24657-63). Therefore, it was investigated whether F16-mediated PTP opening could be the result of F16-induced mitochondrial depolarization. In principle, mitochondrial depolarization could be caused either by a direct protonophoric effect, i.e., by proton shuttling across the inner mitochondrial membrane, or indirectly as a consequence of inhibition of the respiratory chain. First, the effects of F16 were measured on basal (state 4) and uncoupled mitochondrial respiration as follows. Oxygen consumption was determined using a Clarke-type oxygen electrode equipped with thermostatic control and magnetic stirring. Mitochondria (1 mg/ml) were incubated in experimental buffer supplemented with substrates for each respiratory chain complex as indicated in FIG. 7: 5 mM glutamate plus 2.5 mM malate for complex 1, 5 mM succinate-Tris in the presence of 2 μM rotenone for complex II+III, 3 mM ascorbate plus 150 μM TMPD in the presence of 1 μg/ml antimycin A for complex IV.

The results indicate that F16 causes an increase in state 4 of mitochondrial oxygen consumption in mitochondria incubated with substrates for complexes I and II+III. At higher concentrations, this increase droped in a CsA-sensitive fashion. This is due to loss of pyridine nucleotides from the mitochondrial matrix as a consequence of PTP opening, since it is not observed when mitochondria are energized with succinate (which feeds electrons directly to complex II). F16 inhibits uncoupled respiration, and CsA completely abolishes this effect. Thus, the respiratory inhibition is a consequence of PTP opening, with subsequent loss of pyridine nucleotides and cytochrome c release.

It was then determined whether F16 directly causes mitochondrial depolarization. The fluorescent properties of F16 were used for this purpose. It was determined whether the fluorescence of a 1 μM solution of F16 changed in response to mitochondrial energization and depolarization. The results indicate that in response to mitochondrial energization, the fluorescence of the F16 solution increases, whereas membrane potential dissipation by FCCP completely reverts this fluorescence increase. Interestingly, when mitochondria were incubated with 50 μM F16, energization lead to a much smaller fluorescence increase. Preincubation of mitochondria with the PTP inhibitor CsA only partially blunted this effect.

Thus, F16 directly causes mitochondrial depolarization and this depolarization is worsened by the secondary PTP opening. Taken together, these results show that F16 acts as a weak protonophore and that it causes PTP opening by lowering the mitochondrial membrane potential.

Recently, attention has been drawn to mitochondria as potential target of anti-cancer therapy, in particular because of their pivotal involvement in cell death. As the molecular dissection of the apoptotic pathway proceeds, it becomes clearer that the crucial role of mitochondria in propagating the cell death signal. In response to most proapoptotic stimuli, mitochondria release proteins—among them cytochrome c, apoptosis inducing factor, and repressors of caspase inhibitors such as Smac/DIABLO, from their intermembrane space (IMS) to the cytosol or the nucleus to amplify and execute the cell death program. Overexpression of the oncogene bcl-2, and inactivation of the tumor supressor p53 are common traits in several neoplasias, impeding the release of IMS protein such as cytochrome c and conferring resistance to several apoptoitc stimuli. Thus, therapeutic interventions that can selectively target the mitochondria of transformed cells and trigger the apoptotic cascade without relaying on intact upstream apoptosis inducing pathways could represent a specific, low toxicity approach in cancer treatment.

### Example 12

### Neu-Dependent Signaling is Inhibited in F16 Treated Cells

In parallel to the characterization of the effect of F16 on mitochondria homeostasis the effect of F16 on cell signaling was investigated. Control and Neu-overexpressing EpH4-A6 cells were left untreated or treated with 3 μM F16 for 24 and 48 hr. Whole cell lysates were prepared as described above. Samples were immnoblotted with antibodies against Neu, PKB, MAPK, as well as antibodies that react with the phosphorylated forms of the proteins, as described above. Samples were probed with anti-β-actin as loading control, also as described above.

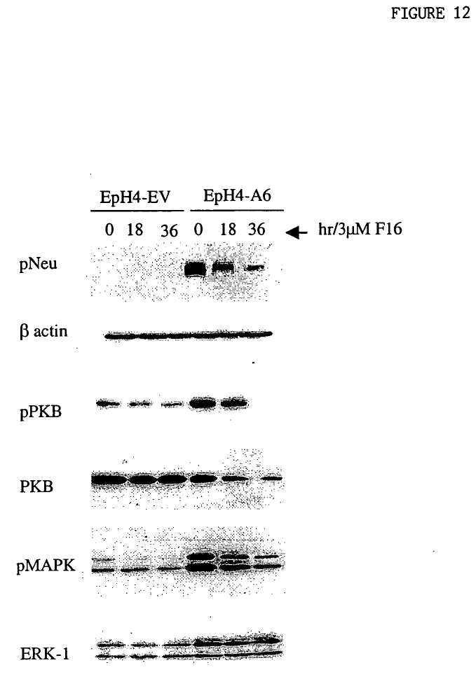

The results (FIG. 12) indicate that the phosphotyrosine content of Neu, phosphorylated PKB/Akt, and phosphorylated MAP kinase are decreased in F16 treated cells. In the case of Neu and PKB, the protein level themselves were reduced as a consequence of F16 treatment. This downregulation was only partially blocked by the pan-caspase inhibitor Z-VAD-FMK. In fact, previous studies have shown that Neu and PKB/Akt are substrates for caspase-mediated cleavage (Tikhomirov et al. J Biol Chem (276) 33675-80 and Bachelder et al. J Biol Chem (276) 34702-7). In addition to caspase activity the observed effects could be the combined result of an increased degradation of damaged proteins and of a decrease in overall protein synthesis. The F16-mediated mitochondrial damage limits the availability of ATP, and protein tranaslation is an energy-dependent process. This in turn may affect the steady-state level of proteins in a differential manner, depending on their particular turn-over rates, since MAPK and actin levels were not affected. Nevertheless, whatever mechanisms account for these findings, the net effect of F16 on cell signaling is inactivation of major pathways involved in proliferation and survival, further contributing to the inhibition of cell growth.

### Example 13

### Identification of QR as a Potent Inhibitor of Cell Proliferation

In testing the growth inhibitory effect of compounds having a chemical structure that is similar to that of F16, a compound was identified that has a strong anti-proliferative effect on transformed cell and only at much lower doses on normal cells. The compound is Quinaldine Red (QR), which is available from Aldrich Sigma under catalog no. 20,131-6, and has the following structure:

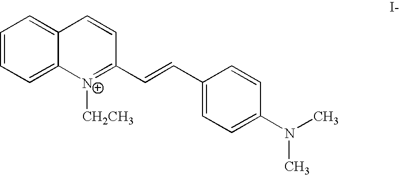

QR is red, absorbs at about 520 nm and emits at about 620 nm, thereby being readily detectable.

The compound was included in cytoblot experiments, as described above, on the following cells: EpH4-A6 and control EpH4-EV cells and the following human breast cancer cell lines: FKBR3 (Neu positive cells); MDA-MD-468 (which over-express EGFR); and MCF-7 (which over-express Her3/ErbB3; EGFR and Neu).

Compound QR was found to inhibit proliferation of all of the transformed cells tested at concentrations as low as 10 nM. Proliferation was essentially completely inhibited at 100 nM, and partially inhibited at 10 nM. Some cells were also killed by the compound. Inhibition of proliferation was essentially specific to transformed cells, since the normal cells were inhibited only at concentrations of 10 μM or higher.

This compound behaves similarly to F16, at least since it was also shown to accumulate in mitochondria of cancer cells and essentially not in mitochondria of normal cells.

Equivalents

Those skilled in the art will recognize, or be able to ascertain using no more than routine experimentation, many equivalents of the specific embodiments of the invention described herein. Such equivalents are intended to be encompassed by the following claims.

1. A pharmaceutical composition comprising one or more compounds having the general structure A, B, C, D, E, F, G, H, I, J or K, or a pharmaceutically acceptable salt or prodrug thereof, and a pharmaceutically acceptable carrier, diluent or excipient.

2. The pharmaceutical composition of claim 1, comprising one or more compounds selected from the group consisting of E20 (ID 141525); F12 (ID 120590); F 16 (ID 274873); H10 (ID 120670); J6 (ID 120856); N12 (ID 215015); L4 (ID 121113); B15 (ID217496) and QR, or derivatives or analogs thereof or a pharmaceutically acceptable salt or prodrug thereof, and a pharmaceutically acceptable carrier, diluent or excipient.

3. The pharmaceutical composition of claim 1, which inhibits cell proliferation and/or modulates differentiation or induces the cell death of target cells.

4. A method for preparing a pharmaceutical composition of claim 1, comprising mixing a growth inhibiting amount or a differentiation stimulating amount or a cell death inducing amount of one or more compounds having the general structure A, B, C, D, E, F, G, H, I, J or K, or a pharmaceutically acceptable salt or prodrug thereof, in a pharmaceutically acceptable carrier, diluent or excipient.

5. A method for inhibiting the proliferation and/or stimulating the differentiation of a cell or inducing cell death of the cell, comprising contacting the cell with an effective amount of one or more compounds having the general structure A, B, C, D, E, F, G, H, I, J or K, or salt or prodrug thereof, such that the proliferation of the transformed cell is inhibited, or its differentiation stimulated or cell death is induced.

6. The method of claim 5, comprising contacting the cell with an effective amount of one or more compounds selected from the group consisting of E20 (ID 141525); F12 (ID 120590); F16 (ID 274873); H10 (ID 120670); J6 (ID 120856); N12 (ID 215015); L4 (ID 121113); B15 (ID217496) and QR, or derivative, analog, salt or prodrug thereof.

7. The method of claim 6, wherein the compound is F16 or QR or prodrug thereof.

8. The method of claim 5, wherein the cell is subject to unwanted proliferation.

9. The method of claim 5, wherein the cell comprises an activated form of a proto-oncogene.

10. The method of claim 9, wherein the cell comprises a Neu or a Ras oncogene.

11. The method of claim 5, wherein the compound is contacted with the cell at a concentration from about 100-500 nM.

12. The method of claim 5, wherein the compound is contacted with the cell at a concentration from about 10-50 nM.

13. Use of one or more compounds having the general structure A, B, C, D, E, F, G, H, I, J or K, or a pharmaceutically acceptable salt or prodrug thereof in an amount effective to inhibit growth of target cells, and a pharmaceutically acceptable carrier, diluent or excipient, for the preparation of a pharmaceutical medicament for inhibiting growth of a target cell in a subject.

14. The use of claim 13, wherein the one or more compounds are selected from the group consisting of E20 (ID 141525); F12 (ID 120590); F16 (ID 274873); H10 (ID 120670); J6 (ID 120856); N12 (ID 215015); L4 (ID 121113); B15 (ID217496) and QR, or derivative, analog, salt or prodrug thereof, or a pharmaceutically acceptable salt thereof.

15. The use of claim 14, wherein the compound is F16 or QR or prodrug thereof.

16. The use of claim 13, wherein the target cell is a cancer cell.

17. The use of claim 13, wherein the target cell expresses an oncogene.

18. The use of claim 17, wherein the oncogene is Neu or Ras.

19. The use of claim 16, wherein the cancer cell is a breast cancer cell.

20. Use of one or more compounds having the general structure A, B, C, D, E, F, G, H, I, J or K, or a pharmaceutically acceptable salt or prodrug thereof in an amount effective to inhibit growth of cancer cells, and a pharmaceutically acceptable carrier, diluent or excipient, for the preparation of a pharmaceutical medicament for treating cancer in a subject.

21. The use of claim 20, wherein the one or more compounds are selected from the group consisting of E20 (ID 141525); F12 (ID 120590); F16 (ID 274873); H10 (ID 120670); J6 (ID 120856); N12 (ID 215015); L4 (ID 121113); B15 (ID 217496) and QR, or derivative, analog, salt or prodrug thereof.

22. The use of claim 21, wherein the compound is F16 or QR or prodrug thereof.

23. The use of claim 20 wherein the cancer is associated with expression of an oncogene.

24. The use of claim 23, wherein the oncogene is the Neu oncogene.

25. The use of claim 20, wherein the cancer is a carcinoma.

26. The use of claim 20, wherein the carcinoma is breast or ovarian carcinoma.
